(19) United States
(12) Patent Application Publication
(10) Pub. No.: US 2024/0277693 A1
(43) Pub. Date: Aug. 22, 2024

(54) A PHARMACEUTICAL PRODUCT CONTAINING TASQUINIMOD AND A METHOD FOR ASSESSING THE PURITY OF SAID PRODUCT

(71) Applicant: ACTIVE BIOTECH AB, Lund (SE)

(72) Inventor: Hans Wännman, Ängelholm (SE)

(73) Assignee: ACTIVE BIOTECH AB, Lund (SE)

(21) Appl. No.: 18/571,472

(22) PCT Filed: Jun. 30, 2022
(86) PCT No.: PCT/EP2022/068063

§ 371 Date: Dec. 18, 2023

(30) Foreign Application Priority Data
Jul. 2, 2021 (EP) 21183481.7

(51) Int. Cl. A61K 31/4704; C07D 215/22

(52) U.S. Cl. CPC A61K 31/4704; C07D 215/22

(57) ABSTRACT

A pharmaceutical composition containing tasquinimod or a pharmaceutically salt of tasquinimod, a method for assessment thereof and a process for its manufacture. The use in therapy of a pharmaceutical composition containing tasquinimod or a pharmaceutically acceptable salt of tasquinimod. The compound 3,3′-methylenebis(4-hydroxy-5-methoxy-1-methylquinoline-2(1H)-one) and its use in a method for assessing a pharmaceutical composition containing tasquinimod or a pharmaceutically acceptable salt of tasquinimod.

### FIELD OF THE INVENTION

The present invention relates to a pharmaceutical product containing tasquinimod or a pharmaceutically acceptable salt of tasquinimod. The invention also relates to a method for the manufacture of such product, and to a method for assessing such product. The invention also relates to a novel compound, useful in particular in such method.

### BACKGROUND OF THE INVENTION

Tasquinimod, or 4-hydroxy-5-methoxy-N, 1-dimethyl-2-oxo-N-[4-(trifluoromethyl)phenyl]-1,2-dihydroquinoline-3-carboxamide, is a compound having the structural formula:

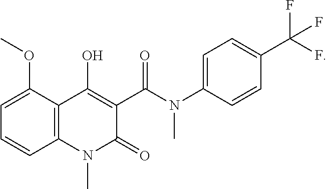 [SMILES: COc1cccc2c1c(O)c(C(=O)N(C)c1ccc(C(F)(F)F)cc1)c(=O)n2C]

Tasquinimod and a method for its preparation were described in international applications No. PCT/SE99/00676, published as WO 99/55678 and No. PCT/SE99/01270, published as WO 00/03991, which applications also disclosed the utility of tasquinimod and some other quinoline carboxamides for the treatment of diseases resulting from autoimmunity, such as multiple sclerosis, insulin-dependent diabetes mellitus, systemic lupus erythematosus, rheumatoid arthritis, inflammatory bowel disease and psoriasis and, furthermore, diseases where pathologic inflammation plays a major role, such as asthma, atherosclerosis, stroke and Alzheimer's disease.

Processes for preparing tasquinimod have also been described in international application No. PCT/SE2003/000780, published as WO 03/106424 and in international application No. PCT/EP2011/061490, published as WO 2012/004338. While these applications disclose processes for preparing quinoline carboxamides, such as tasquinimod, with high yield and high purity, they do not deal with the subsequent preparation and assessment of pharmaceutical compositions containing such quinoline carboxamides as active ingredients, and do not mention any possible degradation of, for example tasquinimod, once incorporated into a pharmaceutical composition.

A deuterated form of tasquinimod and a process for its preparation were described in international application No. PCT/EP2012/061798, published as WO 2012/175541.

A pharmaceutical composition containing tasquinimod particles having high dissolution rate is described in international application No. PCT/EP2022/063887 (not yet published), which also discloses a solid pharmaceutical dosage unit, such as a capsule or tablet for oral administration, containing such particles.

The use of various quinoline carboxamides for the treatment of cancer, more particularly solid cancers, such as prostate cancer and breast cancer, was disclosed in international application No. PCT/SE00/02055, published as WO 01/30758. It has been found that these compounds bind to and inhibit the interactions of an immunomodulatory protein (S100A9), which protein promotes tumor development, influences suppressive and pro-angiogenic cells in the tumor microenvironment and participates in the establishment of pre-metastatic niches.

International application No. PCT/EP2015/075769, published as WO 2016/078921, discloses tasquinimod for use in the treatment of leukemia including acute lymphoblastic leukemia, acute myeloid leukemia, chronic lymphocytic leukemia and chronic myeloid leukemia. International application No. PCT/EP2015/071391, published as WO 2016/042112, discloses tasquinimod for use in the treatment of multiple myeloma. International application No. PCT/EP2016/053288, published as WO 2016/146329, discloses tasquinimod for use in combination with a PD-1 and/or PD-L1 inhibitor in the treatment of cancer, in particular bladder cancer. The use of tasquinimod for the treatment of myeloproliferative neoplasms, such as myelofibrosis, has been disclosed in international application No. PCT/EP2021/070629, published as WO 2022/018240. The use of tasquinimod for the treatment of myelodysplastic syndrome has been described in international application No. PCT/EP2022/050891, not yet published.

The above identified prior art publications are all incorporated herein by reference.

In order to be practically useful in therapy, a therapeutically active agent should normally be provided as a pharmaceutical product suitable for administration to the user, which product must fulfil various quality and safety requirements. Therefore, for any given pharmaceutical product, it is of importance to be able to assess the suitability of the product in terms of, for example, purity and stability, before allowing the product to be used or distributed. Such assessment may include identifying and quantifying degradation and reaction products of the active ingredient in the pharmaceutical product.

### SUMMARY OF THE INVENTION

A first aspect is a pharmaceutical composition comprising a therapeutically effective amount of tasquinimod, or a pharmaceutically acceptable salt thereof, and one or more pharmaceutically acceptable excipients, said pharmaceutical composition further comprising one or more compounds selected from:

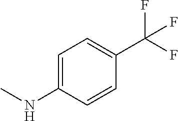

N-methyl-4-(trifluoromethyl)aniline,

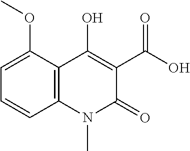 [SMILES: COc1cccc2c1c(O)c(C(=O)O)c(=O)n2C]

4-hydroxy-5-methoxy-1-methyl-2-oxo-1,2-dihydroquinoline-3-carboxylic acid,

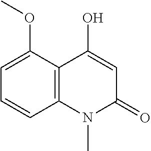

4-hydroxy-5-methoxy-1-methylquinoline-2(1H)-one, and

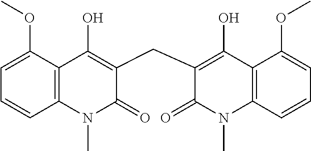 [SMILES: COc1cccc2c1c(O)c(Cc1c(O)c3c(OC)cccc3n(C)c1=O)c(=O)n2C]

3,3′-methylenebis(4-hydroxy-5-methoxy-1-methylquinoline-2(1H)-one),
and pharmaceutically acceptable salts thereof.

A further aspect is a pharmaceutical dose unit for oral administration, comprising the pharmaceutical composition as defined herein.

A further aspect is a pharmaceutical composition or a pharmaceutical dose unit as defined herein, for use in the treatment of cancer.

A further aspect is a method for assessing a pharmaceutical product containing a therapeutically effective amount of tasquinimod or a pharmaceutically acceptable salt thereof, and one or more pharmaceutically acceptable excipients, by obtaining a sample of said product and determining the amount of a tasquinimod decomposition product in said sample, said tasquinimod decomposition product comprising one or more compounds selected from N-methyl-4-(trifluoromethyl)aniline, 4-hydroxy-5-methoxy-1-methyl-2-oxo-1,2-dihydroquinoline-3-carboxylic acid, 4-hydroxy-5-methoxy-1-methylquinoline-2(1H)-one, and 3,3′-methylenebis(4-hydroxy-5-methoxy-1-methylquinoline-2(1H)-one), and pharmaceutically acceptable salts thereof. In some embodiments, the method comprises subjecting a sample of the pharmaceutical product to stability testing followed by determining an amount of said tasquinimod decomposition product in the sample. The method of the invention is useful for assessing a pharmaceutical product containing a therapeutically effective amount of tasquinimod or a pharmaceutically acceptable salt thereof and a pharmaceutically acceptable carrier, e.g. for determining whether the pharmaceutical product is suitable for distribution. In some embodiments, the pharmaceutical product is determined as suitable for distribution only if a sample of the product contains not more than about 5% w/w of said decomposition product, relative to the amount of tasquinimod in the sample.

A further aspect is a process for the manufacture of a pharmaceutical product comprising a pharmaceutically effective amount of tasquinimod or a pharmaceutically acceptable salt thereof, and a pharmaceutically acceptable excipient, said pharmaceutical product further comprising one or more compounds selected from N-methyl-4-(trifluoromethyl)aniline, 4-hydroxy-5-methoxy-1-methyl-2-oxo-1,2-dihydroquinoline-3-carboxylic acid, 4-hydroxy-5-methoxy-1-methylquinoline-2(1H)-one, and 3,3′-methylenebis(4-hydroxy-5-methoxy-1-methylquinoline-2(1H)-one), said process comprising preparing a pharmaceutical composition comprising a pharmaceutically effective amount of tasquinimod or a pharmaceutically acceptable salt thereof and one or more pharmaceutically acceptable excipients, optionally processing the composition to obtain a pharmaceutical dose unit, and assessing the pharmaceutical product by a method as defined herein.

A further aspect is the use of a compound selected from N-methyl-4-(trifluoromethyl)aniline, 4-hydroxy-5-methoxy-1-methyl-2-oxo-1,2-dihydroquinoline-3-carboxylic acid, 4-hydroxy-5-methoxy-1-methylquinoline-2(1H)-one, and 3,3′-methylenebis(4-hydroxy-5-methoxy-1-methylquinoline-2(1H)-one) in a method or a process as defined herein.

A further aspect is the compound 3,3′-methylenebis(4-hydroxy-5-methoxy-1-methylquinolin-2(1H)-one), having the structural formula

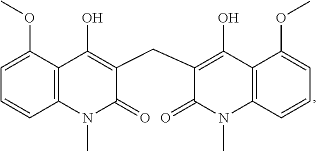 [SMILES: COc1cccc2c1c(O)c(Cc1c(O)c3c(OC)cccc3n(C)c1=O)c(=O)n2C]

or a salt thereof.

A further aspect is the use of the pharmaceutical composition of the invention, in the manufacture of a medicament for the treatment of cancer.

A further aspect is a method for the treatment of cancer, by administering a pharmaceutical composition of the invention or a pharmaceutical dose unit of the invention, to a mammal in need of such treatment.

Further aspects and embodiments thereof will become apparent from the below description.

### BRIEF DESCRIPTION OF THE DRAWINGS

FIG. 1 is an HPLC chromatogram showing the order of elution of compounds I to IV, as defined herein, where ABR-221019 is compound I, ABR-221020 is compound II, ABR-221023 is compound III, and ABR-225865 is compound IV.

FIG. 2 is an ESI mass spectrum of compound IV, prepared in Example 1.

FIG. 3 is a production MS spectrum of m/z 423 of compound IV, prepared in Example 1.

FIG. 4 is a ¹H NMR spectrum of compound IV, prepared in Example 1.

FIG. 5 is a ¹³C NMR spectrum of compound IV, prepared in Example 1.

### DETAILED DESCRIPTION OF THE INVENTION

Unless otherwise indicated or clearly apparent from the context, all technical and scientific terms and abbreviations used herein have the same meaning as commonly understood by one of ordinary skill in the field of art to which this disclosure belongs. However, definitions of some of the terms used herein will be given herein below.

As used herein, and unless otherwise specified or apparent from the context, the term “decomposition product”, or “tasquinimod decomposition product” or “decomposition product as defined herein” etc. refers to one or more compounds derived from the decomposition of tasquinimod in a pharmaceutical composition containing one or more excipients, said compounds being selected from N-methyl-4-(trifluoromethyl)aniline, 4-hydroxy-5-methoxy-1-methyl-2-oxo-1,2-dihydroquinoline-3-carboxylic acid, 4-hydroxy-5-methoxy-1-methylquinoline-2(1H)-one, and 3,3′-methylenebis(4-hydroxy-5-methoxy-1-methylquinoline-2(1H)-one) and pharmaceutically acceptable salts thereof.

As used herein, the term “effective”, means effective to achieve an end or desired object. Thus, for example, “therapeutically effective amount”, refers to a quantity of a component that is sufficient to yield an indicated therapeutic response without undue adverse side effects (such as toxicity, irritation, or allergic response) commensurate with a reasonable benefit/risk ratio when used in the manner of this disclosure. An effective amount of a medicinal agent may vary according to factors, such as the disease state, age, sex, and weight of the human or animal being treated.

The term “excipient” refers to a pharmaceutically acceptable chemical such as known to those of ordinary skill in the art of pharmacy to aid the administration of a medicinal agent. It is a compound that is useful in preparing a pharmaceutical composition, generally safe, non-toxic and neither biologically nor otherwise undesirable, and includes excipients that are acceptable for veterinary use as well as human pharmaceutical use. Exemplary excipients include encapsulating agents, sweeteners, taste-masking agents, carriers, binders, fillers, diluents, disintegrants, anti-adherents, and lubricants.

“Optional” or “optionally” means that the subsequently described event or circumstance may or may not occur, and that the description includes instances where the event or circumstance occurs and instances where it does not.

The term “pharmaceutical dose unit” (or sometimes only “dose unit”) as used herein includes any device useful for administering a given dose of a drug product to a patient, e.g. a capsule, a tablet, a sachet, a micro-capsule, etc.

By “pharmaceutically acceptable” is meant a material that is not biologically or otherwise undesirable, i.e., the material can be administered to an individual along with the relevant active compound without causing clinically unacceptable biological effects or interacting in a deleterious manner with any of the other components of the formulation in which it is contained.

Examples of pharmaceutically acceptable salts comprise salts with (as counter ion) an alkali metal ion, e.g. Li⁺, Na⁺ or K⁺, or with an alkaline earth metal ion, e.g. Mg²⁺ or Ca^{2+,} or with any other pharmaceutically acceptable metal ion, e.g. Zn²⁺ or Al³⁺; or pharmaceutically acceptable salts formed with organic bases, such as diethanolamine, ethanolamine, N-methylglucamine, triethanolamine or tromethamine. Pharmaceutically acceptable salts may also include salts with inorganic or organic acids, such as hydrohalic acids (eg. HCl) or carboxylic acids, e.g. acetic acid, succinic acid, tartaric acid, benzoic acid etc.

It should be noted that tasquinimod as referred to herein may have any degree of deuteration. In some embodiments, tasquinimod has a degree of deuteration corresponding to the natural abundance of the deuterium isotope. In some other embodiments, tasquinimod as used herein is as described in WO 2012/175541, cf. herein above.

Depending on the type of product, which may be e.g. a composition containing tasquinimod for use in a tabletting process or encapsulating process, or the final tablet or capsule, the expression “suitable for distribution” as used herein may mean that the product is suitable for the further processing necessary to obtain a useful drug product, such as an orally administrable capsule or tablet, or that the product is suitable for use by a patient. In other words, a method for assessing a pharmaceutical product may be performed on a pharmaceutical composition containing tasquinimod or a pharmaceutically acceptable salt thereof and a pharmaceutically acceptable carrier, as well as on a pharmaceutical dose unit, such as a tablet or capsule, prepared by use of such composition.

In the present context, the expression “determining that a product is suitable for distribution”, or similar expression as may be used herein, means that, based on the determined amount of a decomposition product as defined herein in a sample of the product, optionally after stability testing, the product is considered to fulfil selected requirements. It should be noted, however, that the product may also have to fulfil other requirements that are not assessed in the method described herein and that are not considered herein. Therefore, in the present context “determining that a product is suitable for distribution” means that the product has been determined to fulfil the selected requirements, e.g. in terms of sufficiently low levels of a tasquinimod decomposition product, but may not necessarily mean that the product fulfils or has been determined to fulfil also every other requirement that may be relevant in the pharmaceutical field.

As used herein, a “pharmaceutical product containing tasquinimod or a pharmaceutically acceptable salt of tasquinimod” (also sometimes referred to herein simply as “pharmaceutical product” or “product”) may be, for example, a pharmaceutical composition containing tasquinimod or a pharmaceutically acceptable salt of tasquinimod in admixture with one or more excipients, or a capsule or tablet for oral administration containing such composition. It is contemplated that a pharmaceutical product containing, as active ingredient, tasquinimod or a pharmaceutically acceptable salt thereof, will contain a therapeutically effective amount of said active ingredient. According to the present invention, it has been found that a pharmaceutical product containing a therapeutically effective amount of tasquinimod or a pharmaceutically acceptable salt thereof as active ingredient also contains a minor amount of a decomposition product as defined herein.

The term “sample” refers to a portion of a product, normally a small portion taken for the purpose of testing the product, e.g. for stability testing of the product. More specifically, in the present context, “a sample” will refer to a sample taken from a pharmaceutical product containing tasquinimod or a pharmaceutically acceptable salt thereof and one or more excipients. The sample may be, for example, a portion of a powder composition, or a number of dose units, such as capsules or tablets, containing a pharmaceutical composition.

It has been found that in particular in the presence of excipients in a pharmaceutical composition, tasquinimod degrades to N-methyl-4-(trifluoromethyl)aniline and 4-hydroxy-5-methoxy-1-methyl-2-oxo-1,2-dihydroquinoline-3-carboxylic acid. The latter may then be decarboxylated to 4-hydroxy-5-methoxy-1-methylquinoline-2(1H)-one. Additionally, a dimer impurity, 3,3′-methylenebis(4-hydroxy-5-methoxy-1-methylquinoline-2(1H)-one has been identified in a pharmaceutical composition containing tasquinimod. In the following, the compounds N-methyl-4-(trifluoromethyl)aniline, 4-hydroxy-5-methoxy-1-methyl-2-oxo-1,2-dihydroquinoline-3-carboxylic acid, 4-hydroxy-5-methoxy-1-methylquinoline-2(1H)-one, and 3,3′-methylenebis(4-hydroxy-5-methoxy-1-methylquinoline-2(1H)-one, may be referred to as compound I, compound II, compound III, and compound IV, respectively.

The chemical names and structural formulas of compounds I-IV are shown in Table 1.

TABLE 1

| Compound | Chemical name | Structural formula |
|---|---|---|
| I | N-methyl-4-(trifluoromethyl)aniline | 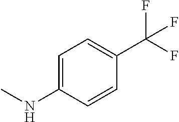 |
| II | 4-hydroxy-5-methoxy-1-methyl-2-oxo-1,2- dihydroquinoline-3-carboxylic acid | 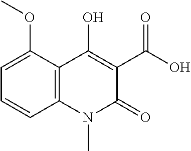 [SMILES: COc1cccc2c1c(O)c(C(=O)O)c(=O)n2C] |
| III | 4-hydroxy-5-methoxy-1-methylquinolin- 2(1H)-one | 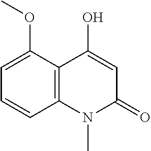 |
| IV | 3,3′-methylenebis(4-hydroxy-5-methoxy-1- methylquinolin-2(1H)-one | 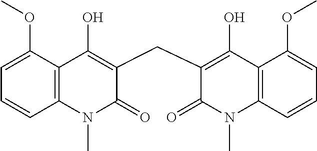 [SMILES: COc1cccc2c1c(O)c(Cc1c(O)c3c(OC)cccc3n(C)c1=O)c(=O)n2C] |

The possible degradation pathways leading to these compounds are illustrated herein below.

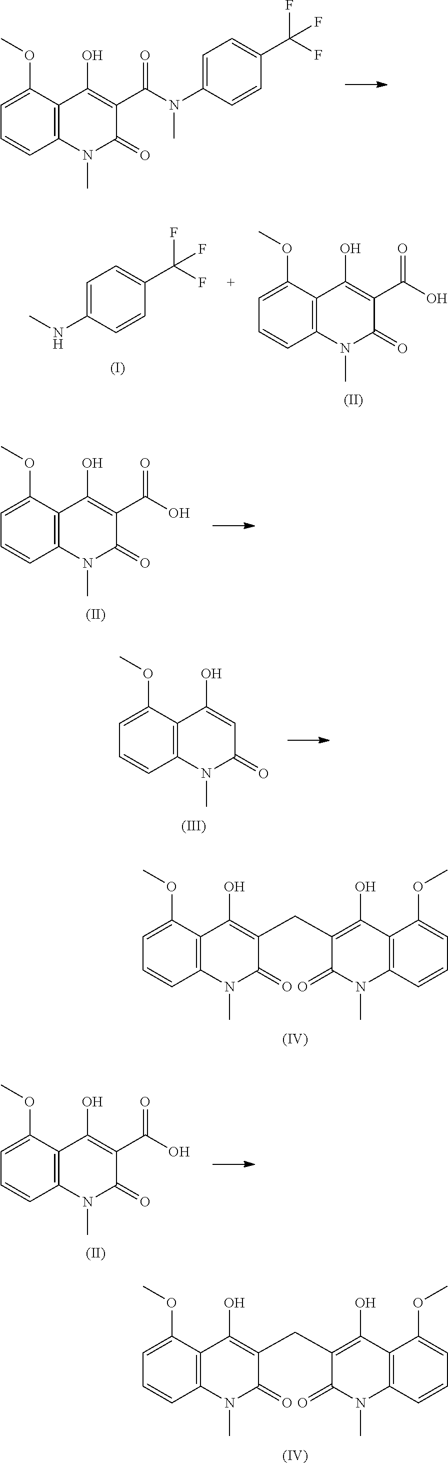

In a drug product, compound I is formed with compound II from hydrolysis of tasquinimod. In drug product stability studies, the amount of compound I was observed to increase under any storage conditions. Compound II is further decarboxylated to form compound III. However, in drug product stability studies a slight increase of compound II was observed under any storage conditions indicating that compound II is formed faster than it is decarboxylated into compound III.

Compound III is the decarboxylated species formed from the degradation of compound II. In drug product stability studies, the amount of compound III was observed to increase under any conditions.

In drug product, compound IV is a dimer formed with two quinoline moieties and its amount was observed to increase under accelerated conditions. The mechanism for formation of compound IV in a drug product is unknown.

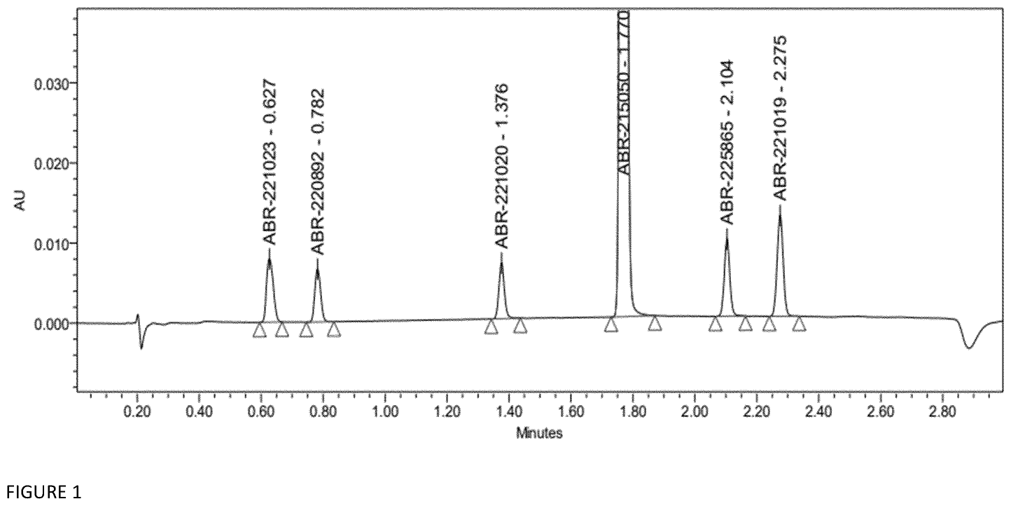

The amounts of compounds I to IV in a tasquinimod drug product have been studied and a chromatogram showing the order of elution of compounds I to IV is shown in FIG. 1.

Table 2 lists the relative response factors used to convert the area % detected into a weight % for each one of compounds I-IV.

TABLE 2

| Compound | Relative response factor |
|---|---|
| I | 1.0 |
| II | 0.7 |
| III | 0.6 |
| IV | 0.6 |

### The Assessment Method

Disclosed herein is a method comprising obtaining a sample of a pharmaceutical product containing tasquinimod or a pharmaceutically acceptable salt thereof and a pharmaceutically acceptable carrier; and determining the amount of a decomposition product of tasquinimod in the sample, said decomposition product comprising one or more compounds selected from compound I, compound II, compound III and compound IV, or a pharmaceutically acceptable salt thereof. Such a method is useful, for example, for assessing a pharmaceutical product containing tasquinimod or a pharmaceutically acceptable salt of tasquinimod in terms of absence of excessive levels of a tasquinimod decomposition product therein.

Thus, in some embodiments, a method for assessing a pharmaceutical product containing tasquinimod or a pharmaceutically acceptable salt of tasquinimod is provided, comprising obtaining a sample of a pharmaceutical product containing tasquinimod or a pharmaceutically acceptable salt thereof and a pharmaceutically acceptable carrier; and determining the amount of a decomposition product of tasquinimod in the sample, said decomposition product comprising one or more compounds selected from compound I, compound II, compound III and compound IV.

It is pointed out that as used herein, and unless otherwise indicated or apparent from the context, the reference to a compound selected from any one of compounds I, II, III, IV may also include a salt thereof, preferably a pharmaceutically acceptable salt.

In some embodiments, a method is provided, comprising obtaining a sample of a pharmaceutical product containing tasquinimod or a pharmaceutically acceptable salt thereof and a pharmaceutically acceptable carrier; keeping (storing) the sample for a period of time;

and determining the amount of a decomposition product of tasquinimod in the sample, said decomposition product comprising one or more compounds selected from compound I, compound II, compound III and compound IV.

Such a method is useful, for example, for assessing a pharmaceutical product containing tasquinimod or a pharmaceutically acceptable salt of tasquinimod, in terms of chemical stability of tasquinimod contained therein.

In some embodiments, a method is provided, comprising obtaining a sample of a pharmaceutical product containing tasquinimod or a pharmaceutically acceptable salt thereof and a pharmaceutically acceptable carrier; optionally keeping the sample for a period of time at a temperature and relative humidity (RH) of the surrounding atmosphere; and determining the amount of a decomposition product of tasquinimod in the sample, said decomposition product comprising one or more compounds selected from compound I, compound II, compound III and compound IV. Such a method may be used for assessing the suitability for distribution of a pharmaceutical product containing tasquinimod or a pharmaceutically acceptable salt of tasquinimod, e.g. in terms of chemical stability of tasquinimod contained therein and/or absence of excessive levels of a tasquinimod decomposition product therein.

In some embodiments the method comprises determining whether a pharmaceutical product containing tasquinimod or a pharmaceutically acceptable salt thereof and a pharmaceutically acceptable carrier is suitable for distribution. In some embodiments, the pharmaceutical product containing tasquinimod or a pharmaceutically acceptable salt of tasquinimod is determined as suitable for distribution only if a sample of said product contains not more than about 5% w/w of a decomposition product of tasquinimod, relative to the amount of tasquinimod of the sample.

In some embodiments, therefore, a method is provided, comprising obtaining a sample of a pharmaceutical product containing tasquinimod or a pharmaceutically acceptable salt thereof and a pharmaceutically acceptable carrier; optionally keeping the sample for a period of time at a temperature and relative humidity of the surrounding atmosphere; and determining whether the amount of a decomposition product of tasquinimod in the sample is not more than about 5% w/w, relative to the amount of tasquinimod in the sample, said decomposition product comprising one or more compounds selected from compound I, compound II, compound III and compound IV.

By “keeping the sample for a period of time at a temperature and relative humidity of the surrounding atmosphere” is meant that the sample is kept (stored) for a period of time in, for example, a closed container, an open container or in a suitable packaging (e.g. a blister package) at a temperature, which may be a specific temperature or a temperature range, e.g. room temperature (about 18-25° C.), or higher than room temperature (about 25-45° C.), and a relative humidity of, for example 30 to 50%, or higher, e.g. 60 to 75% RH. The period of time may be, for example, weeks, months or years, e.g. 2 weeks to 5 years. Keeping or storing a sample for a period of time at a temperature and relative humidity may also be referred to herein as “stability testing”.

In view of the above, in some embodiments, a method is provided for assessing a pharmaceutical product containing tasquinimod or a pharmaceutically acceptable salt thereof and a pharmaceutically acceptable excipient, said method comprising obtaining a sample of the product, subjecting the sample to stability testing, and determining the amount of a tasquinimod decomposition product in the sample at the end of the stability testing, said tasquinimod decomposition product comprising one or more compounds selected from compound I, compound II, compound III, and compound IV, or a pharmaceutically acceptable salt of any one of these compounds.

In some embodiments, a method is provided for assessing a pharmaceutical product containing tasquinimod or a pharmaceutically acceptable salt thereof and a pharmaceutically acceptable excipient, said method comprising obtaining at least two samples of the product, determining an amount of a tasquinimod decomposition product in one or more of the samples of the product before any stability testing, subjecting the other sample or samples of the product to stability testing, followed by determining an amount of a tasquinimod decomposition product in the other sample or samples, said tasquinimod decomposition product comprising one or more compounds selected from compound I, compound II, compound III, and compound IV, or a pharmaceutically acceptable salt of any one of these compounds. Such a method may comprise comparing, the determined amounts of a tasquinimod decomposition product in the different samples, to assess the pharmaceutical product in terms of chemical stability of tasquinimod therein.

In some embodiments, the method may include obtaining several samples of the product and submitting the several samples to stability testing for different lengths of time, e.g. 2 weeks, 1 month, 3 months, 6 months etc, and determining an amount of a tasquinimod decomposition product in samples having undergone stability testings of different length in time.

In some embodiment, the “pharmaceutical product” referred to herein is a batch of the product, and the method as described herein may be used to assess the quality of the batch, e.g. in terms of amount of tasquinimod in the batch, compared to the target amount of tasquinimod (e.g. the intended dose strength of a dose unit), chemical stability of tasquinimod in the batch, and/or amount of a tasquinimod decomposition product in the batch.

In some embodiments, the method for assessing a pharmaceutical product containing tasquinimod or a pharmaceutically acceptable salt of tasquinimod is a method for determining whether a product is suitable for distribution. In some embodiments, the method for assessing the pharmaceutical product is part of a method for determining whether a product is suitable for distribution, which method may also include determining whether other criteria are fulfilled, e.g. absence of visible defects in a dose unit (such as cracks or miscolouring), absence of microbial contamination, and, for a powder composition, may include determining whether the composition has suitable pharmacotechnical properties, such as flowability, bulk density, tapped density, Carr's Index, water content etc. Thus, in some cases, even if the pharmaceutical product is deemed suitable for distribution according to a criterion for assessment as described herein, it may still happen that the product is not deemed suitable for distribution for other reasons, not taken into consideration by the present method. Therefore, in the present context “suitable for distribution” may refer to a situation where the product is deemed suitable for distribution in accordance with the results of the method described herein, including situations where the product is deemed unsuitable for distribution for some other reason.

In some embodiments, the product is deemed suitable for distribution only if a sample thereof contains not more than about 5% w/w of a tasquinimod decomposition product as defined herein, relative to the amount of tasquinimod in the sample. In some of these embodiments, the sample may not contain more than about 2% w/w, relative to tasquinimod, of any one of compounds I, II, III and IV.

In some embodiments, therefore, the method comprises determining whether a sample of a pharmaceutical product containing tasquinimod or a pharmaceutically acceptable salt of tasquinimod, optionally at the end of stability testing, contains not more than about 5% w/w, relative to tasquinimod, of a tasquinimod decomposition product comprising one or more compounds selected from compound I, compound II, compound III, and compound IV, and pharmaceutically acceptable salts thereof, and determining whether the sample contains not more than about 2% w/w of any one of these compounds (i.e. none of the compounds is present in an amount of more than about 2% w/w).

In some embodiments, the method comprises determining whether the sample, optionally at the end of stability testing, contains not more than about 2% w/w, relative to tasquinimod, of a tasquinimod decomposition product comprising one or more compounds selected from compound I, compound II, compound III, and compound IV, and pharmaceutically acceptable salts thereof, and determining whether the sample contains not more than about 0.5% w/w of any one of these compounds.

In some embodiments, the pharmaceutical product is a pharmaceutical composition containing tasquinimod or a pharmaceutically acceptable salt of tasquinimod in admixture with one or more excipients, and a sample of said composition can be, for example, 100 mg to 100 g of said composition.

In some embodiments, the pharmaceutical product is a pharmaceutical dose unit, e.g. a capsule or tablet for oral administration containing tasquinimod in admixture with one or more excipients, and a sample of said product may be one such dose unit or a number of such dose units, e.g. 1 to 100 capsules or tablets, or 1 to 50 capsules or tablets, or 1 to 20 capsules or tablets, or 1 to 10 capsules or tablets, or 1 to 5 capsules or tablets, e.g. at least 2, at least 3, at least 5, at least 10, or at least 20 capsules or tablets.

In some embodiments, the method disclosed herein comprises storing (keeping) a sample of the pharmaceutical product for a period of time, preferably under selected conditions of temperature and humidity. The amount (or concentration) of tasquinimod and of at least one of compounds I, II, III and IV in the sample may preferably be determined at the beginning or before the storage period and at the end of the storage period. The selected storage conditions may include a temperature in the range of, for example, 20° C. to 40° C., and a relative humidity in the surrounding atmosphere in the range of, for example, 30% to 75%; e.g. 25° C. and 60% RH, or 30° C. and 65% RH, or 35° C. and 70% RH, or 40° C. and 75% RH. The storage period may extend over a time period in the range of, for example, several weeks to several years. For example, the stability testing period (“storage period”) may be 2 weeks, 3 weeks, 1 month, 2 months, 3 months, 4 months, 5 months, 6 months, 9 months, 1 year, 18 months, 2 years, 3 years, 4 years or even 5 years or longer. A shorter storage period may be selected by use of higher storage temperature and/or higher relative humidity (RH). The total amount of a tasquinimod decomposition product in the sample at the end of the storage period may be determined by, for example, HPLC.

For stability testing, the sample may be stored or kept in a closed container or in an open container. In some embodiments, e.g. when the pharmaceutical product is a dose unit, such as a capsule or tablet, the sample may be stored in a packaging such as a blister package or a medicinal glass or plastic jar or vial.

The method disclosed herein includes determining the amount of one or more compounds selected from compound I, compound II, compound III, and compound IV in a sample of the product. In some embodiments, the method includes determining the amount of compound I, and optionally the amount of one or more of compounds II, III, and IV. In some embodiments, the method includes determining the amounts of compounds I and II, and optionally the amount of one or both of compounds III and IV. In some embodiments, the method includes determining the amounts of compounds I, II, and III, and optionally the amount of compound IV. In some embodiments, the method includes determining the amounts of compounds I, II, III, and IV.

In some further embodiments, the method includes determining the amount of compound II, and optionally the amount of one or more of compound I, compound III, and compound IV in the sample.

In some further embodiments, the method includes determining the amount of compound II and compound III, and optionally the amount of one or both of compound I and compound IV in the sample.

In some further embodiments, the method includes determining the amount of compound II, compound III, and compound IV, and optionally the amount of compound I in the sample.

In some further embodiments, the method includes determining the amount of compound III, and optionally the amount of one or more of compound I, compound II, and compound IV in the sample.

In some further embodiments, the method includes determining the amount of compound III and compound IV, and optionally the amount of one or both of compound I, and compound II in the sample.

In some further embodiments, the method includes determining the amount of compound IV, and optionally the amount of one more of compound I, compound II, and compound IV in the sample.

In some further embodiments, the method includes determining the amount of compound I in the sample. In some further embodiments, the method includes determining the amount of compound II in the sample. In some further embodiments, the method includes determining the amount of compound III in the sample. In some further embodiments, the method includes determining the amount of compound IV in the sample.

In some embodiments of a method for determining whether a pharmaceutical product containing tasquinimod is suitable for distribution, the product is determined as being suitable for distribution only if a sample of the product, optionally after stability testing (i.e. at the end of a selected stability testing period), contains not more than about 5% w/w of a tasquinimod decomposition product as defined herein above. Determining that a product is suitable for distribution may also be referred to herein as “approving the product for distribution”. In some embodiments, the method includes approving the product for distribution only if the sample thereof contains not more than about 4.5% w/w of a tasquinimod decomposition product as defined herein, or not more than: about 4.0% w/w, about 3.5% w/w, about 3.0 w/w, about 2.5% w/w about 2.0% w/w, about 1.5% w/w, about 1.0% w/w, 0.8% w/w, about 0.7% w/w, about 0.6% w/w, or about 0.5% w/w of said tasquinimod decomposition product, relative to the amount of tasquinimod in the sample.

In some embodiments, the method comprises approving the product for distribution only if a sample of the product, optionally after stability testing, contains not more than about 2.0% w/w of compound I, not more than about 1.5% w/w of compound I, not more than about 1.0% w/w of compound I, not more than 0.50% w/w of compound I, or not more than about 0.25% w/w of compound I, relative to the amount of tasquinimod in the sample.

In some embodiments, the method comprises approving the product for distribution only if a sample of the product, optionally after stability testing, contains not more than about 2.0% w/w of compound II, not more than about 1.5% w/w of compound II, not more than about 1.0% w/w of compound II, not more than 0.50% w/w of compound II, or not more than about 0.25% w/w of compound II, relative to the amount of tasquinimod in the sample.

In some embodiments, the method comprises approving the product for distribution only if a sample of the product, optionally after stability testing, contains not more than about 2.0% w/w of compound III, not more than about 1.5% w/w of compound III, not more than about 1.0% w/w of compound III, not more than 0.50% w/w of compound III, or not more than about 0.25% w/w of compound III, relative to the amount of tasquinimod in the sample.

In some embodiments, the method comprises approving the product for distribution only if a sample of the product, optionally after stability testing, contains not more than about 2.0% w/w of compound IV, not more than about 1.5% w/w of compound IV, not more than about 1.0% w/w of compound IV, not more than 0.50% w/w of compound IV, or not more than about 0.25% w/w of compound IV, relative to the amount of tasquinimod in the sample.

In some embodiments, the method comprises approving the product for distribution only if a sample of the product, optionally after stability testing, contains (a) not more than about 5% w/w of a tasquinimod decomposition product, as defined herein above; and (b) not more than about 2.0% w/w of any one of compounds I-IV, not more than about 1.5% w/w of any one of compounds I-IV, not more than about 1.0% w/w of any one of compounds I-IV, not more than 0.50% w/w of any one of compounds I-IV, or not more than about 0.25% w/w of any one of compounds I-IV, relative to the amount of tasquinimod in the sample.

In some embodiments, the method comprises approving the product for distribution only if a sample of the product, optionally after stability testing, contains (a) not more than about 4% w/w of a tasquinimod decomposition product, as defined herein above; and (b) not more than about 2.0% w/w of any one of compounds I-IV, not more than about 1.5% w/w of any one of compounds I-IV, not more than about 1.0% w/w of any one of compounds I-IV, not more than 0.50% w/w of any one of compounds I-IV, or not more than about 0.25% w/w of any one of compounds I-IV, relative to the amount of tasquinimod in the sample.

In some embodiments, the method comprises approving the product for distribution only if a sample of the product, optionally after stability testing, contains (a) not more than about 3% w/w of a tasquinimod decomposition product, as defined herein above; and (b) not more than about 1.5% w/w of any one of compounds I-IV, not more than about 1.0% w/w of any one of compounds I-IV, not more than 0.50% w/w of any one of compounds I-IV, or not more than about 0.25% w/w of any one of compounds I-IV, relative to the amount of tasquinimod in the sample.

In some embodiments, the method comprises approving the product for distribution only if a sample of the product, optionally after stability testing, contains (a) not more than about 2% w/w of a tasquinimod decomposition product, as defined herein above; and (b) not more than about 1.0% w/w of any one of compounds I-IV, not more than 0.50% w/w of any one of compounds I-IV, or not more than about 0.25% w/w of any one of compounds I-IV, relative to the amount of tasquinimod in the sample.

In some embodiments, the method comprises approving the product for distribution only if a sample of the product, optionally after stability testing, contains (a) not more than about 5% w/w of a tasquinimod decomposition product, as defined herein above; and (b) not more than about 2.0% w/w of compound I, not more than about 1.5% w/w of compound I, not more than about 1.0% w/w of compound I, not more than 0.50% w/w of compound I, or not more than about 0.25% w/w of compound I, relative to the amount of tasquinimod in the sample; and/or not more than about 2.0% w/w of compound II, not more than about 1.5% w/w of compound II, not more than about 1.0% w/w of compound II, not more than 0.50% w/w of compound II, or not more than about 0.25% w/w of compound II, relative to the amount of tasquinimod in the sample; and/or not more than about 2.0% w/w of compound III, not more than about 1.5% w/w of compound III, not more than about 1.0% w/w of compound III, not more than 0.50% w/w of compound III, or not more than about 0.25% w/w of compound III, relative to the amount of tasquinimod in the sample; and/or not more than about 2.0% w/w of compound IV, not more than about 1.5% w/w of compound IV, not more than about 1.0% w/w of compound IV, not more than 0.50% w/w of compound IV, or not more than about 0.25% w/w of compound IV, relative to the amount of tasquinimod in the sample.

In some embodiments, the method further comprises determining the amount of tasquinimod in a sample of a pharmaceutical product, relative to a target amount of tasquinimod in the product, which may be the required dose strength of tasquinimod in a pharmaceutical dose unit, such as 1 mg of tasquinimod/dose unit, or the corresponding amount of a pharmaceutically acceptable salt of tasquinimod. For example, in some embodiments, the method comprises storing samples of the pharmaceutical product for a period of time, periodically determining the amount of tasquinimod in a sample, and comparing the determined amount of tasquinimod in the sample with a target amount of tasquinimod, which may be, for example, the amount of tasquinimod present in a dose unit at the beginning of the storage period, or the intended dose strength.

### The Process for Preparing a Product

A further aspect is a process for preparing a pharmaceutical product comprising a therapeutically effective amount of tasquinimod or a pharmaceutically acceptable salt of tasquinimod and a pharmaceutically acceptable excipient, said process including a method as described herein for assessing the pharmaceutical product.

In some embodiments, for example, a process is provided for preparing a pharmaceutical product comprising tasquinimod or a pharmaceutically acceptable salt of tasquinimod, said process comprising: admixing tasquinimod or a pharmaceutically acceptable salt thereof with one or more excipients to obtain a pharmaceutical composition, optionally subjecting the pharmaceutical composition to additional processing, e.g. to obtain a dosage unit, such as a tablet or capsule, obtaining a sample of the product, optionally subjecting the sample to stability testing, and determining the amount of a tasquinimod decomposition product in the sample, said tasquinimod decomposition product comprising one or more compounds selected from N-methyl-4-(trifluoromethyl)aniline, 4-hydroxy-5-methoxy-1-methyl-2-oxo-1,2-dihydroquinoline-3-carboxylic acid, 4-hydroxy-5-methoxy-1-methylquinolin-2(1H)-one, and 3,3′-methylenebis(4-hydroxy-5-methoxy-1-methylquinolin-2(1H)-one).

In some embodiments, said process comprises determining whether a sample of the product contains not more than about 5% w/w, relative to the amount of tasquinimod in the sample, of a decomposition product as defined herein and approving the pharmaceutical product for distribution if the sample contains not more than about 5% w/w of the decomposition product, relative to the amount of tasquinimod in the sample.

In some preferred embodiments, said process comprises determining the amount of a decomposition product as defined herein in a sample of the pharmaceutical product containing tasquinimod, and approving the product for distribution only if the sample contains no more than about 2% w/w of the decomposition product, relative to the amount of tasquinimod in the sample.

The process for preparing a pharmaceutical product comprising tasquinimod may include a process for synthesizing and isolating tasquinimod, or a pharmaceutically acceptable salt thereof, e.g. as described in any of the above-mentioned publications, e.g. WO 03/106424 and WO 2012/004338, followed by combining tasquinimod, or the pharmaceutically acceptable salt thereof, with one or more suitable, pharmaceutically acceptable excipients, to obtain a pharmaceutical composition containing tasquinimod or a pharmaceutically acceptable salt of tasquinimod, optionally followed by preparing a suitable pharmaceutical dose unit containing said composition, such as an oral capsule or an oral tablet.

Thus, the method for assessment as disclosed herein may be part of a process for preparing a pharmaceutical product as defined herein. For example, a process for preparing a pharmaceutical dose unit for oral administration containing tasquinimod or a pharmaceutically acceptable salt of tasquinimod, e.g. a capsule or a tablet, may comprise preparing the dose unit by a conventional encapsulation or tabletting of a pharmaceutical composition containing tasquinimod or a pharmaceutically acceptable salt of tasquinimod, and submitting a sample of the obtained capsules or tablets to an assessment method as described herein, to verify that the tablets or capsules comply with requirements in terms of maximum level of an undesired tasquinimod reaction or decomposition product as defined herein.

A process for preparing a dose unit, such as a capsule or tablet, containing a therapeutically effective amount of tasquinimod may comprise blending tasquinimod or a pharmaceutically acceptable salt of tasquinimod with a filler, admixing a lubricating agent with the obtained blend, processing the obtained composition into dose units, e.g. by tabletting or encapsulating the composition, determining an amount of a tasquinimod decomposition product as defined herein in a sample of said dose units, and optionally storing a sample of said dose units for a period of time, e.g. 2 weeks to 5 years, or 1 month to 5 years, or 1 month to 4 years, or 1 month to 3 years, or 1 month to 2 years, or 1 month to 1 year, or 1 month to 3 months, e.g. 3 months to 5 years, or 3 months to 4 years, or 3 months to 3 years, or 3 months to 2 years, or 3 months to 1 year, or 6 months to 5 years, or 6 months to 4 years, or 6 months to 3 years, or 6 months to 3 years, or 6 months to 2 years, or 6 months to 1 year, at a temperature of 25 ° ° C. to 40° C., and 60% to 75% RH, and determining an amount of a tasquinimod decomposition product as defined herein in the sample at the end of the storage period.

In some embodiments, multiple samples are obtained from one and the same pharmaceutical product and each sample is independently subjected to stability testing, e.g. for different lengths of time and/or at different conditions of temperature and relative humidity, whereby the amount of a tasquinimod decomposition product as defined herein is determined in each sample separately.

### The Pharmaceutical Product

Also provided herein is a solid pharmaceutical product comprising a therapeutically effective amount of tasquinimod or a pharmaceutically acceptable salt thereof as an active ingredient, a pharmaceutically acceptable excipient, and additionally comprising a tasquinimod decomposition product comprising one or more compounds selected from compound I, compound II, compound III, and compound IV, or pharmaceutically acceptable salts of compounds I, II, III and IV. Preferably, in such a product, said tasquinimod decomposition product is present in an amount of not more than about 5% w/w relative to the amount of tasquinimod.

In such a product, tasquinimod may be present in free base form or as a pharmaceutically acceptable salt. Examples of pharmaceutically acceptable salts comprise salts with (as counter ion) an alkali metal ion, e.g. Li⁺, Na⁺ or K⁺, or with an alkaline earth metal ion, e.g. Mg²⁺ or Ca²⁺, or with any other pharmaceutically acceptable metal ion, e.g. Zn²⁺ or Al³⁺; or pharmaceutically acceptable salts formed with organic bases, such as diethanolamine, ethanolamine, N-methylglucamine, triethanolamine or tromethamine.

Such salts, as well as e.g. acid addition salts, e.g. with strong acids such as hydrohalic acids, may also be formed by the decomposition products of tasquinimod.

Herein, therefore, both “tasquinimod” and “tasquinimod decomposition product” should be understood to include the free base form as well as the salt form of said compounds, unless otherwise indicated or apparent from the context. In connection to this, it is pointed out that any weight and % w/w indicated herein, of either a tasquinimod decomposition product or of tasquinimod should be understood to refer to the non-salt (free base) form of the tasquinimod decomposition product and of tasquinimod.

In some embodiments, the pharmaceutical product contains not more than about 4.5% w/w, relative to tasquinimod, of a tasquinimod decomposition product, as defined herein, e.g. not more than about 4.0% w/w, 3.5% w/w, 3.0% w/w, 2.5% w/w, 2.0% w/w, 1.5% w/w, 1.0% w/w, 0.8% w/w, 0.7% w/w, 0.6% w/w, or 0.5% w/w of a tasquinimod decomposition product as defined herein, relative to the amount of tasquinimod in the product.

It goes without saying that it is preferred that as low an amount as possible of a tasquinimod decomposition product is present in the product of the invention. However, in some embodiments, the pharmaceutical product contains at least 0.01% w/w, relative to tasquinimod, of a tasquinimod decomposition product as defined herein above, e.g. at least 0.02% w/w, 0.03% w/w, 0.04% w/w, 0.05% w/w, 0.06% w/w, 0.07% w/w, 0.08% w/w, 0.09% w/w, or 0.10% w/w relative to the amount of tasquinimod in the product, of a tasquinimod decomposition product as defined herein above.

In some embodiments, the pharmaceutical product is a composition containing a therapeutically effective amount of tasquinimod, and one or more pharmaceutically acceptable excipients, including a filler and/or a lubricant. In some embodiments, the composition is a particle composition, or comprises a powder, e.g. as described in international application No. PCT/EP2022/063887 (cf. above).

For example, the pharmaceutical product may be a composition containing about 0.1 to 2% by weight of tasquinimod, and about 98 to 99.9% by weight of one or more pharmaceutically acceptable excipients, e.g. about 0.2 to 1% by weight of tasquinimod, and about 99 to 99.8% by weight of one or more pharmaceutically acceptable excipients.

In some embodiments, the pharmaceutical product is a composition containing a therapeutically effective amount of tasquinimod and one or more pharmaceutically acceptable excipients, e.g. tasquinimod, a filler and a lubricant.

In some embodiments, the pharmaceutical product is a composition containing tasquinimod and one or more pharmaceutically acceptable excipients, e.g. tasquinimod, pregelatinised starch as a filler, and a hydrogenated vegetable oil as a lubricant.

In some embodiments, the pharmaceutical product is a pharmaceutical dose unit, e.g. a pharmaceutical dose unit for oral administration, such as a capsule or tablet. For example, the pharmaceutical product may be a dose unit containing from 0.25 mg to 2 mg of tasquinimod, e.g. from 0.5 to 1.5 mg of tasquinimod, in particular 1 mg of tasquinimod (or the corresponding amount of a pharmaceutically acceptable salt thereof), and one or more pharmaceutically acceptable excipients, such as a filler and a lubricant.

In some embodiments, the pharmaceutical dose unit is a tablet for oral administration. In some other embodiments, the pharmaceutical dose unit is a capsule for oral administration.

For example, the pharmaceutical product may be a capsule for oral administration, such as a hard-shell capsule, the capsule containing from 0.25 mg to 2 mg tasquinimod, e.g. from 0.5 to 1.5 mg of tasquinimod, in particular 1 mg of tasquinimod (or the corresponding amount of a pharmaceutically acceptable salt thereof), and one or more pharmaceutically acceptable excipients, such as a filler and a lubricant.

In some embodiments, the pharmaceutical product is a solid, immediate release capsule containing about 100 to about 200 mg, e.g. about 150 mg, of a powder composed of from 0.1 mg to 2 mg of tasquinimod, in combination with one or more pharmaceutically acceptable excipients, e.g. pregelatinised starch as a filler and hydrogenated vegetable oil as a lubricant.

As noted herein above, in a pharmaceutical product determined as suitable for distribution, as provided herein, compounds I-IV are present in a total amount of not more than about 5% w/w, relative to the amount of tasquinimod in the product. It should be realized that in some cases, the amount of compounds I-IV in a pharmaceutical product containing tasquinimod may be very low, e.g. close to or even under the detection limit.

In some embodiments, at least one of compounds I-IV is present in the pharmaceutical product in an amount of at least about 0.01% w/w relative to the amount of tasquinimod in the product, at least about 0.02% w/w, at least about 0.05% w/w, at least about 0.08% w/w, or at least about 0.10% w/w, relative to the amount of tasquinimod in the product.

In some embodiments, a pharmaceutical product is provided comprising tasquinimod or a pharmaceutically acceptable salt thereof, a pharmaceutically acceptable excipient, and compound I, wherein compound I is present in the pharmaceutical composition in an amount of not more than about 2.0% w/w relative to the amount of tasquinimod, or not more than about 1.5% w/w, about 1.0% w/w, about 0.50% w/w, or about 0.25% w/w relative to the amount of tasquinimod. In some of these embodiments, compound I is present in an amount of at least about 0.01% w/w relative to the amount of tasquinimod in the product, at least about 0.02% w/w, at least about 0.05% w/w, at least about 0.08% w/w, or at least about 0.10% w/w, relative to the amount of tasquinimod in the product.

In some embodiments, a pharmaceutical product is provided comprising tasquinimod or a pharmaceutically acceptable salt thereof, a pharmaceutically acceptable excipient, and compound II, wherein compound II is present in the pharmaceutical composition in an amount of not more than about 2.0% w/w relative to the amount of tasquinimod, or not more than about 1.5% w/w, about 1.0% w/w, about 0.50% w/w, or about 0.25% w/w relative to the amount of tasquinimod. In some of these embodiments, compound II is present in an amount of at least about 0.01% w/w relative to the amount of tasquinimod in the product, at least about 0.02% w/w, at least about 0.05% w/w, at least about 0.08% w/w, or at least about 0.10% w/w, relative to the amount of tasquinimod in the product.

In some embodiments, a pharmaceutical product is provided comprising tasquinimod or a pharmaceutically acceptable salt thereof, a pharmaceutically acceptable excipient, and compound III, wherein compound III is present in the pharmaceutical composition in an amount of not more than about 2.0% w/w relative to the amount of tasquinimod, or not more than about 1.5% w/w, about 1.0% w/w, about 0.50% w/w, or about 0.25% w/w relative to the amount of tasquinimod. In some of these embodiments, compound III is present in an amount of at least about 0.01% w/w relative to the amount of tasquinimod in the product, at least about 0.02% w/w, at least about 0.05% w/w, at least about 0.08% w/w, or at least about 0.10% w/w, relative to the amount of tasquinimod in the product.

In some embodiments, a pharmaceutical product is provided comprising tasquinimod or a pharmaceutically acceptable salt thereof, a pharmaceutically acceptable excipient, and compound IV, wherein compound IV is present in the pharmaceutical composition in an amount of not more than about 2.0% w/w relative to the amount of tasquinimod, or not more than about 1.5% w/w, about 1.0% w/w, about 0.50% w/w, or about 0.25% w/w relative to the amount of tasquinimod. In some of these embodiments, compound IV is present in an amount of at least about 0.01% w/w relative to the amount of tasquinimod in the product, at least about 0.02% w/w, at least about 0.05% w/w, at least about 0.08% w/w, or at least about 0.10% w/w, relative to the amount of tasquinimod in the product.

The pharmaceutical product provided herein (e.g. a pharmaceutical composition or a pharmaceutical dose unit comprising such a pharmaceutical composition) preferably is in solid form. In some embodiments, the pharmaceutical product is a pharmaceutical composition. In some embodiments, the pharmaceutical product is a pharmaceutical dose unit, e.g. an oral capsule or tablet.

It is pointed out that tasquinimod as well as compounds I to IV may exist either in non-salt form or in salt form. Unless otherwise indicated or clearly apparent from the context, any reference to tasquinimod or any one of compounds I to IV should be understood to include the non-salt form as well as the salt form thereof, but any amounts indicated herein refer to the non-salt form of tasquinimod and of compounds I to IV.

### The Use of the Pharmaceutical Product

As mentioned herein above, the therapeutic activity of tasquinimod in the treatment of various diseases has been previously shown. It is considered that the pharmaceutical product provided herein will be useful in therapy, in particular in the treatment of any of those diseases for which tasquinimod has been previously shown as having a therapeutic activity. Thus, a further aspect is the pharmaceutical product provided herein, for use in the treatment of cancer.

A further aspect is the use of a pharmaceutical composition as provided herein in the manufacture of a medicament for the treatment of cancer. In some embodiments, the manufacture comprises preparing a capsule, by encapsulating a pharmaceutical composition as provided herein, using encapsulating techniques well-known in the technical field. In some other embodiments, the manufacture comprises preparing a tablet, using tabletting techniques, also well-known in the technical field. It is noted that the selection of suitable excipients and manufacturing conditions are considered well within the knowledge of the person of ordinary skill in the art, having due regard to the present description and by reference to well-known textbooks such as “Aulton's Pharmaceutics, The Design and Manufacture of Medicines”, 6th Edition 2021, Editors: Kevin Taylor, Michael Aulton (Paperback ISBN: 9780702081545, eBook ISBN: 9780702081569), Copyright: @ Elsevier; or

“Remington, The Science and Practice of Pharmacy”, 23rd Edition 2020, Editor: Adeboye Adejare, (Hardcover ISBN: 9780128200070, eBook ISBN: 9780128223895), Copyright: @ Academic Press.

A still further aspect is a method for the treatment of cancer by administering an effective amount of the pharmaceutical product provided herein to a mammal in need of such treatment. Preferably, the method comprises oral administration of a pharmaceutical dose unit as provided herein, such as an oral tablet or capsule. In embodiments, the method comprises oral administration of a tablet as provided herein. In embodiments, the method comprises oral administration of a capsule as provided herein.

In some embodiments, the cancer is selected from bladder cancer, melanoma, lung cancer such as NSCLC (Non-Small Cell Lung Cancer), colorectal cancer, breast cancer, pancreatic cancer, prostate cancer, renal cell carcinoma, hematologic malignancies, in particular advanced hematologic malignancies, ovarian cancer, in particular, platinum-resistant ovarian cancer, neuroendocrine tumors (NET) and gastroenteropancreatic neuroendocrine tumors (GEP-NET). The cancer treated with the composition of the present invention can be any stage, e.g. early stage or late stage. In some embodiments, the treatment results in sustained response in the individual after cessation of the treatment. In some embodiments, the treatment produces a complete response, a partial response, or stable disease in the individual.

In some embodiments, the cancer is a hematologic cancer, such as leukemia, lymphoma, myelodysplastic syndrome, myeloproliferative neoplasm, or multiple myeloma. In some embodiments, the hematological cancer is selected from leukemia and multiple myeloma. In some embodiments, the hematological cancer is selected from leukemia, myelodysplastic syndrome, and myeloproliferative neoplasm.

In some embodiments, the hematologic cancer is leukemia. In some embodiments, the hematologic cancer is lymphoma. In some embodiments, the hematologic cancer is myelodysplastic syndrome. In some embodiments, the hematologic cancer is a myeloproliferative neoplasm. In some embodiments, the hematologic cancer is multiple myeloma.

The leukemia may be selected from chronic lymphocytic leukemia, including hairy cell leukemia, chronic myeloid leukemia, acute lymphocytic leukemia, and acute myeloid leukemia and its precursor, myelodysplastic syndrome. In some embodiments, the leukemia is acute lymphocytic leukemia, or acute myeloid leukemia and its precursor, myelodysplastic syndrome. In some embodiments, the leukemia is acute lymphocytic leukemia. In some embodiments, the leukemia is acute myeloid leukemia.

In some embodiments, the myeloproliferative neoplasm is selected from the group consisting of myelofibrosis, essential thrombocythemia (ET), polycythemia vera (PV), chronic neutrophilic leukemia, chronic myelogenous leukemia, acute myelogenous leukemia, chronic eosinophilic leukemia and mastocytosis. In some embodiments, the myeloproliferative neoplasm is selected from the group consisting of myelofibrosis, essential thrombocythemia, polycythemia vera, chronic neutrophilic leukemia, chronic eosinophilic leukemia and mastocytosis. In some embodiments, the myeloproliferative neoplasm is selected from the group consisting of myelofibrosis, essential thrombocythemia, and polycythemia vera. In some embodiments, the myeloproliferative neoplasm is myelofibrosis. In some embodiments, the myeloproliferative neoplasm is essential thrombocythemia or polycythemia vera.

Essential thrombocythemia and polycythemia vera can both develop into myelofibrosis. Therefore, in some embodiments, the pharmaceutical product provided herein is for use to prevent or reduce the progression into fibrotic phase of a myeloproliferative neoplasm such as essential thrombocythemia or polycythemia vera. Therefore, the term “myelofibrosis” as used herein refers to primary myelofibrosis, as well as secondary myelofibrosis, including post-ET myelofibrosis, and post-PV myelofibrosis. In some embodiments, the myelofibrosis is primary myelofibrosis. In some embodiments, the myelofibrosis is secondary myelofibrosis.

In some further embodiments, the cancer is a solid cancer, e.g. bladder cancer, prostate cancer or breast cancer. In some embodiments, the cancer is selected from bladder cancer (such as particular non muscle invasive bladder cancer, muscle invasive bladder cancer and metastatic and urothelial bladder cancer), prostate cancer and renal cell carcinoma. In some embodiments, the cancer is bladder cancer.

In the medical treatment of any given subject by use of the pharmaceutical product provided herein, the dosage level and frequency will generally be as determined by the treating physician, with due regard to factors such as sex, age, corporal weight and relative health of the treated subject, the selected route and form of administration, the additional use of other drugs, e.g. in a combination therapy.

Generally, a daily dosage ranging from a minimum of 0.001 mg/kg body weight, or 0.002 mg/kg body weight or 0.005 mg/kg body weight or 0.01 mg/kg body weight, to a maximum of 0.2 mg/kg body weight, or 0.1 mg/kg body weight, or 0.05 mg/kg body weight, or 0.02 mg/kg body weight is contemplated. In some embodiments, tasquinimod is administered in an amount of 0.1 to 4 mg/day, or 0.2 to 2 mg/day, 0.4 to 1.8 mg/day, 0.5 to 1.5 mg/day, or 0.6 to 1.2 mg/day, e.g. 1 mg/day.

In some embodiments, the dosage may be gradually adjusted to reach optimal results, so-called dosage titration. For example, dosage titration may comprise starting with a low daily dosage of e.g. 0.25 mg and maintaining this dose level for a period of 1 or 2 weeks. In case no significant side effects are encountered that may contraindicate raising the dose, the level may then be increased, e.g. to 0.5 mg/day for 1 or 2 weeks, after which period another increase may be contemplated, to reach a daily dosage of 1 mg, and so on. In such a method, if any significant side effects occur after an incremental increase of the dosage, the dosage may again be reduced to a previous level. Side effects that may occur include those that may generally be encountered in this type of treatment, e.g. gastrointestinal problems, tiredness, and flu-like syndrome, considered to be related to dosage.

Tasquinimod preferably is administrated on a daily basis, e.g. 1-3 times a day, or 1-2 times a day, such as once daily. However, in some embodiments, the drug is administrated on a less frequent basis, e.g. every two days, once a week etc. It should be noted that if a pharmaceutically acceptable salt of tasquinimod is administered, an equivalent dosage would be one resulting in the indicated dosage of tasquinimod in non-salt form (i.e. as a free base).

The Compound 3,3′-methylenebis(4-hydroxy-5-methoxy-1-methylquinolin-2(1H)-one)

A further aspect is the compound 3,3′-methylenebis(4-hydroxy-5-methoxy-1-methylquinolin-2(1H)-one), (compound IV), of structural formula:

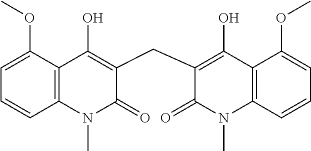 [SMILES: COc1cccc2c1c(O)c(Cc1c(O)c3c(OC)cccc3n(C)c1=O)c(=O)n2C]

or a pharmaceutically acceptable salt of said compound.

Compound IV is useful, for example, in an assessment method as described herein.

A process for Preparing 3,3′-methylenebis(4-hydroxy-5-methoxy-1-methylquinolin-2(1H)-one)

A further aspect is a process for preparing compound IV or a salt of said compound, said process comprising allowing (e.g. by slurrying) 4-hydroxy-5-methoxy-1-methylquinolin-2(1H)-one (compound III) to react with paraformaldehyde in a solvent such as dry (99%) ethanol, in the presence of ethane-1,2-diamine and acetic acid.

In some embodiments, the process comprises obtaining compound III by submitting 4-hydroxy-5-methoxy-1-methyl-2-oxo-1,2-dihydroquinoline-3-carboxylic acid (compound II) to a decarboxylation reaction.

In some embodiments, the process comprises obtaining compound II by hydrolysis of the corresponding C1-C6 alkyl ester, or C1-C3 alkyl ester, e.g. methyl 4-hydroxy-5-methoxy-1-methyl-2-oxo-1,2-dihydroquinoline-3-carboxylate.

In some embodiments, the process comprises transforming the obtained compound IV to a salt thereof, e.g. an alkali metal salt, such as a salt with a metal selected from lithium, sodium, potassium etc.

### The Use of a Compound Selected From Compounds I, II, III and IV

Compounds I, II, III, and IV are useful in methods as disclosed herein, for example, as analytical reference samples or to prepare a calibration curve, e.g. in a method for determining whether a pharmaceutical product containing tasquinimod or a pharmaceutically acceptable salt of tasquinimod is suitable for distribution. Some aspects therefore are directed to the use of any one or more of compounds I, II, III and IV, in a method as disclosed herein, and in process for preparing a pharmaceutical composition comprising tasquinimod wherein such method is applied.

In some embodiments, the use of compound I is provided, in a method as described herein.

In some embodiments, the use of compound II is provided, in a method as described herein. In some embodiments, the use of compound III is provided, in a method as described herein. In some embodiments, the use of compound IV is provided, in a method as described herein.

### EXAMPLES

The invention is further illustrated by the following non-limiting Examples.

### Example 1

### Synthesis of 3,3′-methylenebis(4-hydroxy-5-methoxy-1-methylquinolin-2(1H)-one)

Step 1: 4-Hydroxy-5-methoxy-1-methyl-2-oxo-1,2-dihydroquinoline-3-carboxylic Acid

Methyl 4-hydroxy-5-methoxy-1-methyl-2-oxo-1,2-dihydroquinoline-3-carboxylate (40 g) was hydroyzed with NaCl/H₂SO₄ in acetic acid to give 4-hydroxy-5-methoxy-1-methyl-2-oxo-1,2-dihydroquinoline-3-carboxylic acid (compound II) (23.7 g).

Step 2: 4-Hydroxy-5-methoxy-1-methylquinolin-2(1H)-one

Compound II (12 g) was heated at 120° C. in DMSO for 2 hours, to give 4-hydroxy-5-methoxy-1-methylquinolin-2(1H)-one (compound III) (9.31 g, Mw 205.22).

Step 3: 3,3′-methylenebis(4-hydroxy-5-methoxy-1-methylquinolin-2(1H)-one)

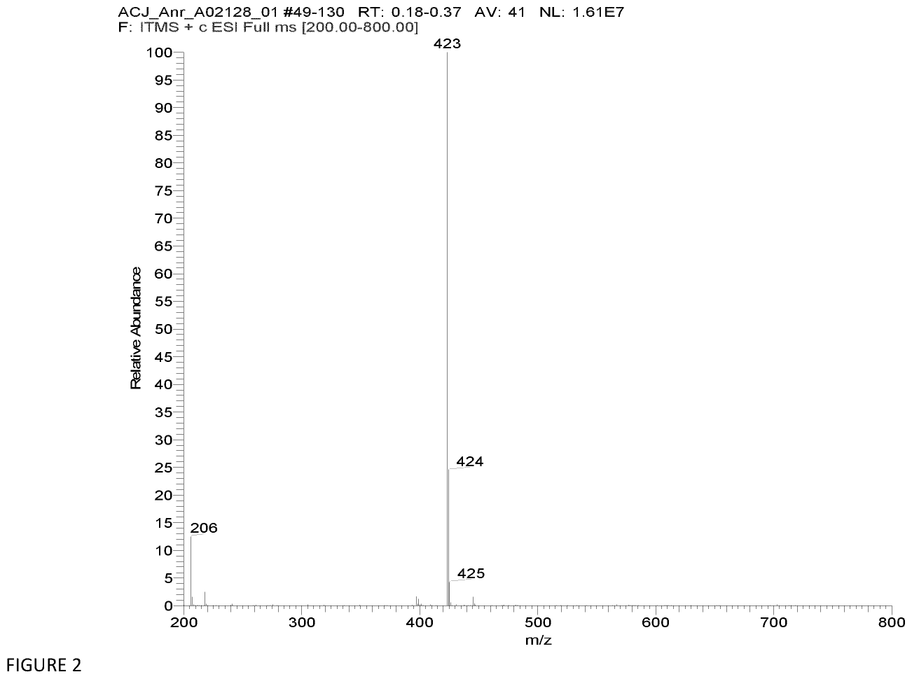

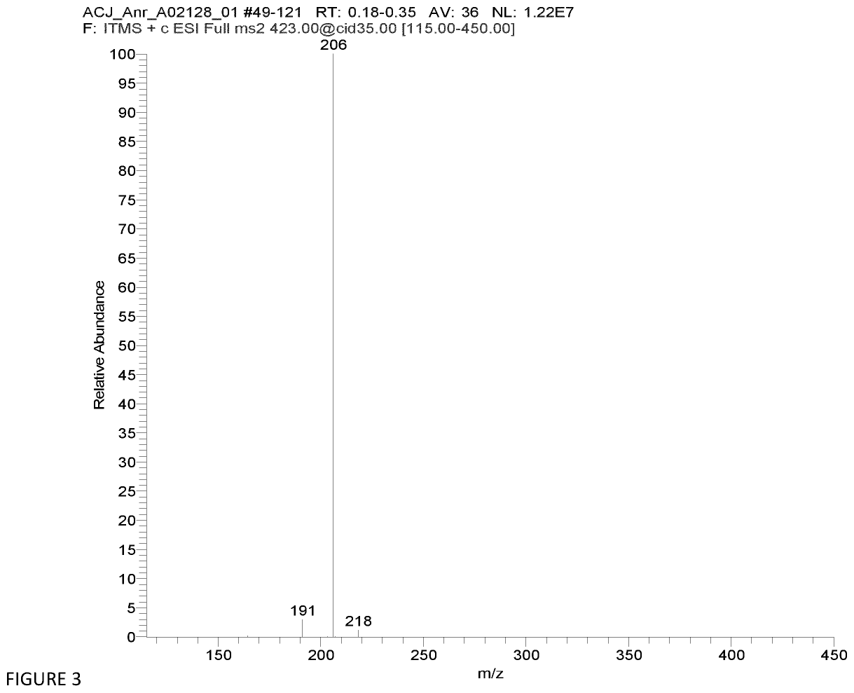

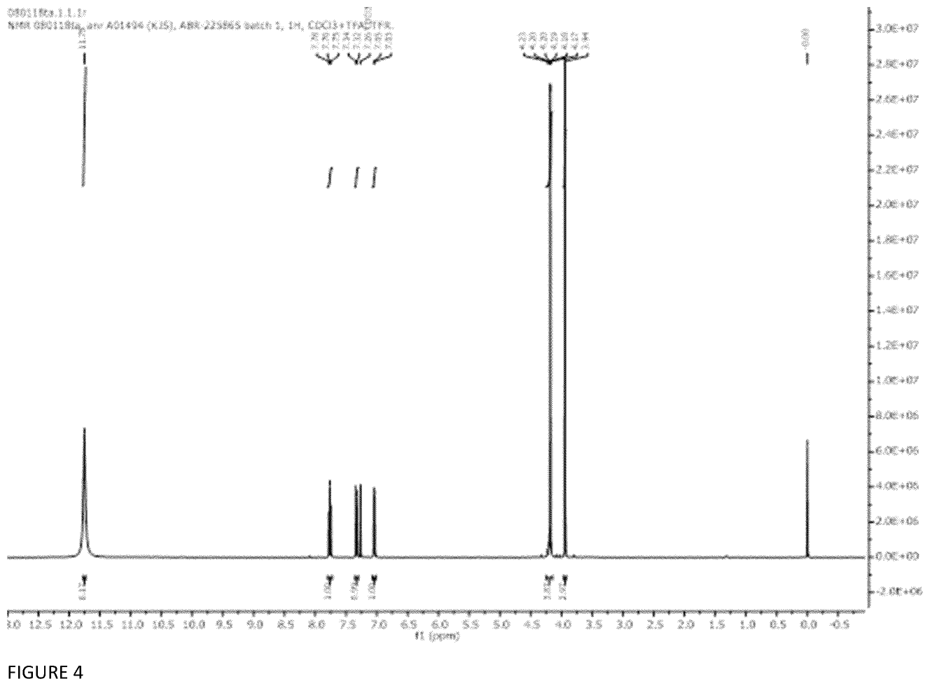

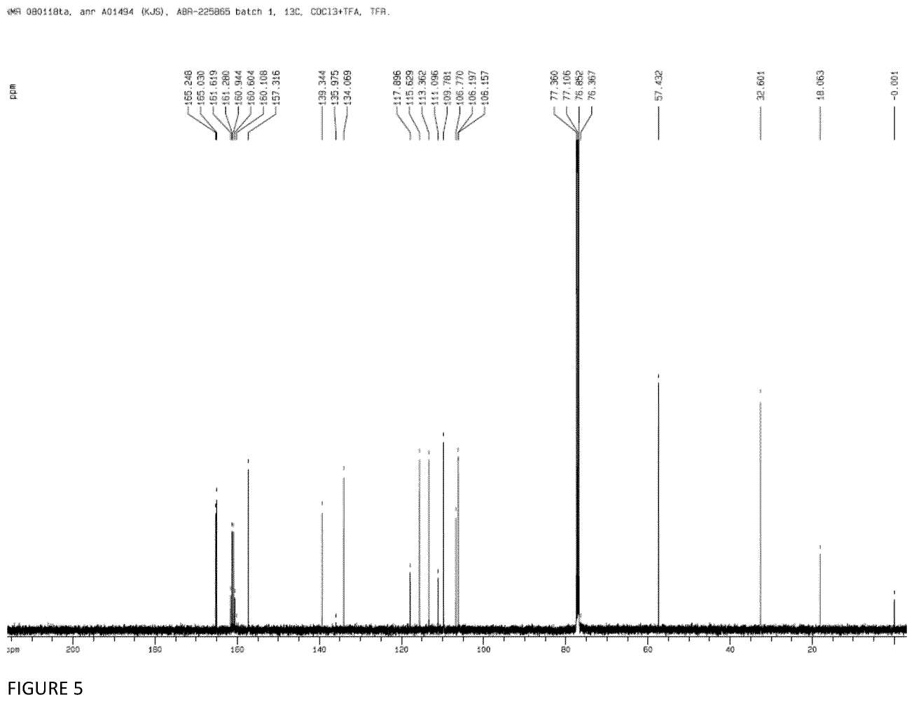

Compound III (3.0 g, 14.6 mmol) and paraformaldehyde (205 mg, 6.1 mmol, 90%) were slurried in 30 ml of dry (99%) ethanol. To the slurry, ethane-1,2-diamine (21 μl) and acetic acid (80 μl) were added and the reaction mixture was refluxed. After 2 hours, the reaction mixture was diluted by addition of EtOH and was filtered, to give 3,3′-methylenebis(4-hydroxy-5-methoxy-1-methylquinolin-2(1H)-one) (compound IV) (2.6 g, Mw 422.43). The structure of compound IV was confirmed by MS, ¹H NMR and ¹³C NMR. Spectra are displayed in FIG. 2, FIG. 3, FIG. 4, and FIG. 5.

### Example 2

### Manufacture and Assessment of Oral Capsules Containing 1 mg of Tasquinimod

A batch of oral capsules having a target dose strength of 1 mg of tasquinimod/capsule was prepared. The capsules were conventional, solid, immediate release capsules (hard gelatin, size 3 Coni-snap® capsules), each capsule containing 154 mg of a powder composed of tasquinimod (target weight 1 mg), pregelatinised starch from maize (Starch 1500®) (150 mg) and hydrogenated vegetable oil (Sterotex®) (3 mg). The manufacturing process involved a single-step blending procedure, followed by filling the required amount of the blend into the capsule shells.

The obtained batch of capsules was assessed by determining the amount of tasquinimod and the amount of compound III in a sample of the capsules. The selected criterion for approving the batch was that the capsules of the sample contain not more than 1.0% w/w of compound III relative to the amount of tasquinimod. The capsules of the sample were found to contain 0.01% w/w of compound III, relative to the amount of tasquinimod (which was determined to be 1.05 mg/capsule). Therefore, according to the selected criterion for approval, the batch of capsules was determined to be suitable for distribution.

### Example 3

### Assessment of Oral Capsules Containing 1 mg of Tasquinimod

The batch of capsules manufactured in Example 2 was assessed by determining the amount of tasquinimod and the amount of compound III in a sample of the capsules, whereby the criterion for approving the batch was that a sample of capsules from the batch, at the end of a 1-month stability testing, contain not more than 1.0% w/w of compound III relative to the amount of tasquinimod. The stability testing consisted of keeping the capsules at 40° C./75% RH in open brown glass jars. At the end of the 1-month stability testing, the capsules contained 0.13% w/w of compound III, relative to the amount of tasquinimod (1.04 mg/capsule). Therefore, according to the selected criterion for approval, the batch of capsules was determined to be suitable for distribution.

### Example 4

### Assessment of Oral Capsules Containing 1 mg of Tasquinimod

The batch of capsules manufactured in Example 2 was assessed by a method as described in Example 3, except for keeping the capsules at 40° C./75% RH in open brown glass jars for 2 months. The selected criterion for approving the batch was that, at the end of the stability test, the capsules of the sample contain not more than 1.0% w/w of compound III relative to the amount of tasquinimod. At the end of the 2-month stability testing, the capsules contained 0.12% w/w of compound III, relative to the amount of tasquinimod (1.04 mg/capsule). Therefore, according to the selected criterion for approval, the batch of capsules was determined to be suitable for distribution.

### Example 5

### Assessment of Oral Capsules Containing 1 mg of Tasquinimod

The batch of capsules manufactured in Example 2 was assessed by a method as described in Example 3, except for keeping the capsules at 40° C./75% RH in open brown glass jars for 3 months. The selected criterion for approving the batch was that, at the end of the stability test, the capsules of the sample contain not more than 1.0% w/w of compound III relative to the amount of tasquinimod. At the end of the 3-month stability testing, the capsules contained 0.14% w/w of compound III, relative to the amount of tasquinimod (1.00 mg/capsule). Therefore, according to the selected criterion for approval, the batch of capsules was determined to be suitable for distribution.

### Example 6

### Assessment of Oral Capsules Containing 1 mg of Tasquinimod

The batch of capsules manufactured in Example 2 was assessed by a method as described in Example 3, except for keeping the capsules at 40° C./75% RH in open brown glass jars for 6 months. The selected criterion for approving the batch was that, at the end of the stability test, the capsules of the sample contain not more than 1.0% w/w of compound III relative to the amount of tasquinimod. At the end of the 6-month stability testing, the capsules contained 0.16% w/w of compound III, relative to the amount of tasquinimod (1.01 mg/capsule). Therefore, according to the selected criterion for approval, the batch of capsules was determined to be suitable for distribution.

### Example 7

### Manufacture and Assessment of Oral Capsules Containing 0.5 mg of Tasquinimod

A batch of oral capsules (about 8000 capsules) having a target dose strength of 0.5 mg of tasquinimod/capsule was prepared. The capsules were conventional, solid, immediate release capsules (hard gelatin, size 3 (3 ml) Coni-snap® capsules), each capsule containing 210 mg of a powder composed of tasquinimod (target weight 0.5 mg), pregelatinised starch from maize (Starch 1500®) (205.3 mg) and hydrogenated vegetable oil (Lubritab®) (4.2 mg). The manufacturing process involved a three-step blending procedure, viz. step 1) a first pre-mixing step comprising mixing tasquinimod with a small part of the filler in a small blender; step 2), a second pre-mixing step after addition of the lubricant and a second fraction of the filler; and step 3) a step of final mixing with the remaining filler in a tumble blender followed by filling the required amount of the blend into the capsule shells.

The obtained batch of capsules was assessed by determining the amount of tasquinimod and the amount of compound III in a sample of the capsules. The selected criterion for approving the batch was that the capsules of the sample contain not more than 1.0% w/w of compound III relative to the amount of tasquinimod. The capsules of the sample were found to contain 0.03% w/w of compound III, relative to the amount of tasquinimod (which was determined to be 0.498 mg/capsule). Therefore, according to the selected criterion for approval, the batch of capsules was determined to be suitable for distribution.

### Example 8

### Assessment of Oral Capsules Containing 0.5 mg of Tasquinimod

The batch of capsules manufactured in Example 7 was assessed by determining the amount of tasquinimod and the amount of compound III in a sample of the capsules, whereby the criterion for approving the batch was that a sample of capsules from the batch, at the end of a 3-month stability testing, contain not more than 1.0% w/w of compound III relative to the amount of tasquinimod in the capsules. The stability testing consisted of keeping the capsules at 40° C./75% RH in sealed brown glass jars. At the end of the stability testing, the capsules contained 0.08% w/w of compound III, relative to the amount of tasquinimod (0.493 mg/capsule). Therefore, according to the selected criterion for approval, the batch of capsules was determined to be suitable for distribution.

### Example 9

### Assessment of Oral Capsules Containing 0.5 mg of Tasquinimod

The batch of capsules manufactured in Example 7 was assessed by a method as described in Example 8, except for keeping the capsules at 40° C./75% RH in sealed brown glass jars for 6 months. At the end of the 6-month stability testing, the capsules contained 0.14% w/w of compound III, relative to the amount of tasquinimod (0.487 mg/capsule). Therefore, according to the selected criterion for approval, the batch of capsules was determined to be suitable for distribution.

### Example 10

### Manufacture and Assessment of Oral Capsules Containing 0.25 mg of Tasquinimod

A batch of oral capsules (6000 capsules) having a target dose strength of 0.25 mg of tasquinimod/capsule was prepared. The capsules were conventional, solid, immediate release capsules (hard gelatin, size 4 Coni-snap® capsules), each capsule containing 150 mg of a powder composed of tasquinimod (target weight 0.25 mg), pregelatinised starch from maize (Starch 1500®) (146.75 mg) and hydrogenated vegetable oil (Lubritab®) (3.00 mg). The manufacturing process involved a three-step blending procedure as described in Example 7, followed by filling the required amount of the blend into the capsule shells.

The obtained batch of capsules was assessed by determining the amount of tasquinimod and the amount of compound III in a sample of the capsules. The selected criterion for approving the batch was that the capsules of the sample contain not more than 1.0% w/w of compound III relative to the amount of tasquinimod. The capsules of the sample were found to contain 0.07% w/w of compound III, relative to the amount of tasquinimod (which was determined to be 0.252 mg/capsule). Therefore, according to the selected criterion for approval, the batch of capsules was determined to be suitable for distribution.

### Example 11

### Assessment of Oral Capsules Containing 0.25 mg of Tasquinimod

The batch of capsules manufactured in Example 10 was assessed by determining the amount of tasquinimod and the amount of compound III in a sample of the capsules, whereby the criterion for approving the batch was that a sample of capsules from the batch, at the end of a 3-month stability testing, contain not more than 1.0% w/w of compound III relative to the amount of tasquinimod in the capsules. The stability testing consisted of keeping the capsules at 40° C./75% RH in sealed brown glass jars. At the end of the stability testing, the capsules contained 0.10% w/w of compound III, relative to the amount of tasquinimod (determined to be 0.253 mg/capsule). Therefore, according to the selected criterion for approval, the batch of capsules was determined to be suitable for distribution.

### Example 12

### Assessment of Oral Capsules Containing 0.25 mg of Tasquinimod

The batch of capsules manufactured in Example 10 was assessed by a method as described in Example 11, except for keeping the capsules at 40° C./75% RH in sealed brown glass jars for 6 months, whereby the criterion for approving the batch was that the capsules of the sample, at the end of the 6-month stability testing, contain not more than 1.0% w/w of compound III relative to the amount of tasquinimod in the capsules. At the end of the 6-month stability testing, the capsules contained 0.10% w/w of compound III, relative to the amount of tasquinimod (determined to be 0.247 mg/capsule). Therefore, according to the selected criterion for approval, the batch of capsules was determined to be suitable for distribution.

### Example 13

### Manufacture and Assessment of Oral Capsules Containing 0.25 mg of Tasquinimod

A batch of oral capsules (80000 capsules) having a target dose strength of 0.25 mg of tasquinimod/capsule was prepared. The capsules were conventional, solid, immediate release capsules (white, hard gelatin, size 4 Coni-snap® capsules), each capsule containing 150 mg of a powder composed of tasquinimod (target weight 0.25 mg), pregelatinised starch from maize (Starch 1500®) (146.75 mg) and hydrogenated vegetable oil (Lubritab®) (3.00 mg). The manufacturing process involved a three-step blending procedure of the same type as described in Example 7, followed by filling the required amount of the blend into the capsule shells.

The obtained batch of capsules was assessed by determining the amount of tasquinimod and the amount of compound III in a sample of the capsules. The selected criterion for approving the batch was that the capsules of the sample contain not more than 1.0% w/w of compound III relative to the amount of tasquinimod. The capsules of the sample were found to contain 0.06% w/w of compound III, relative to the amount of tasquinimod (which was determined to be 0.256 mg/capsule). Therefore, according to the selected criterion for approval, the batch of capsules was determined to be suitable for distribution.

### Example 14

### Assessment of Oral Capsules Containing 0.25 mg of Tasquinimod

The batch of capsules manufactured in Example 13 was assessed by determining the amount of tasquinimod and the amount of compound III in a sample of the capsules, whereby the criterion for approving the batch was that the capsules of the sample, at the end of a 3-month stability testing, contain not more than 1.0% w/w of compound III relative to the amount of tasquinimod in the capsules. The stability testing consisted of keeping the capsules, in a transparent, conventional blister pack, at 40° C./75% RH. At the end of the stability testing, the capsules contained 0.12% w/w of compound III, relative to the amount of tasquinimod (determined to be 0.253 mg/capsule). Therefore, according to the selected criterion for approval, the batch of capsules was determined to be suitable for distribution.

### Example 15

### Assessment of Oral Capsules Containing 0.25 mg of Tasquinimod

The batch of capsules manufactured in Example 13 was assessed by a method as described in Example 14, except for keeping the blister pack with the capsules at 40° C./75% RH for 6 months, whereby the criterion for approving the batch was that the capsules of the sample, at the end of the 6-month stability testing, contain not more than 1.0% w/w of compound III relative to the amount of tasquinimod in the capsules. At the end of the 6-month stability testing, the capsules contained 0.24% w/w of compound III, relative to the amount of tasquinimod (0.247 mg/capsule). Therefore, according to the selected criterion for approval, the batch of capsules was determined to be suitable for distribution.

### Example 16

### Manufacture and Assessment of Oral Capsules Containing 1 mg of Tasquinimod

The general procedure of Example 13 was repeated to manufacture capsules containing 1.00 mg of tasquinimod, in combination with 146.00 mg of Starch 1500®, and 3.00 mg of Lubritab® (i.e. a fill weight of 150 mg/capsule).

The obtained batch of capsules was assessed by determining the amount of tasquinimod and the amount of compound III in a sample of the capsules. The selected criterion for approving the batch was that the capsules of the sample contain not more than 1.0% w/w of compound III relative to the amount of tasquinimod. The capsules of the sample were found to contain 0.02% w/w of compound III, relative to the amount of tasquinimod (which was determined to be 0.994 mg/capsule). Therefore, according to the selected criterion for approval, the batch of capsules was determined to be suitable for distribution.

### Example 17

### Assessment of Oral Capsules Containing 1 mg of Tasquinimod

The batch of capsules manufactured in Example 16 was assessed by determining the amount of tasquinimod and the amount of compound III in a sample of the capsules, whereby the criterion for approving the batch was that the capsules of the sample, at the end of a 3-month stability testing, contain not more than 1.0% w/w of compound III relative to the amount of tasquinimod in the capsules. The stability testing consisted of keeping the capsules, in a transparent, conventional blister pack, at 40° C./75% RH. At the end of the stability testing, the capsules contained 0.13% w/w of compound III, relative to the amount of tasquinimod (determined to be 0.993 mg/capsule). Therefore, according to the selected criterion for approval, the batch of capsules was determined to be suitable for distribution.

### Example 18

### Assessment of Oral Capsules Containing 1 mg of Tasquinimod

The batch of capsules manufactured in Example 16 was assessed by a method as described in Example 14, except for keeping the blister pack with the capsules at 40° C./75% RH for 6 months, whereby the criterion for approving the batch was that the capsules of the sample, at the end of the 6-month stability testing, contain not more than 1.0% w/w of compound III relative to the amount of tasquinimod in the capsules. At the end of the 6-month stability testing, the capsules contained 0.21% w/w of compound III, relative to the amount of tasquinimod (determined to be 0.985 mg/capsule). Therefore, according to the selected criterion for approval, the batch of capsules was determined to be suitable for distribution.

### Example 19

### Manufacture and Assessment of Oral Capsules Containing 0.25 mg of Tasquinimod

A batch of oral capsules (200000 capsules) having a target dose strength of 0.25 mg of tasquinimod/capsule was prepared. The capsules were conventional, solid, immediate release capsules (white, size 4 hard gelatin shell capsules), each capsule containing 150 mg of a powder composed of tasquinimod (target weight 0.25 mg), pregelatinised starch from maize (Starch 1500®) (146.75 mg) and hydrogenated vegetable oil (Lubritab®) (3.00 mg). The manufacturing process involved a three-step blending procedure as described in Example 7, followed by filling the required amount of the blend into the capsule shells.

The obtained batch of capsules was assessed by determining the amount of tasquinimod and the amount of compounds I, II, III and IV in a sample of the capsules. The selected criterion for approving the batch was that the capsules of the sample contain not more than in total 5% w/w of compounds I-IV relative to the amount of tasquinimod. The capsules of the sample were found to contain a total amount of compounds I, II, III and IV of less than 0.2% w/w, relative to the amount of tasquinimod (which was determined to be 0.244 mg/capsule). Therefore, according to the selected criterion for approval, the batch of capsules was determined to be suitable for distribution.

### Example 20

### Assessment of Oral Capsules Containing 0.25 mg of Tasquinimod

The batch of capsules manufactured in Example 19 was assessed by determining the amount of tasquinimod and the amount of each one of compounds I, II, III and IV in a sample of the capsules at the end of a 3-month stability testing, whereby the criterion for approving the batch was that the capsules of the sample, at the end of the 3-month stability testing, contain a total amount of compounds I-IV of not more than 5.0% w/w, relative to the amount of tasquinimod in the capsules. The stability testing consisted of keeping the capsules, in a water impermeable blister pack, at 25° C./60% RH. At the end of the stability testing, the capsules contained 0.06% w/w of compound I, less than 0.05% w/w of compound II, 0.10% of compound III and less than 0.05% w/w of compound IV, relative to the amount of tasquinimod (determined to be 0.246 mg/capsule), viz. in total less than 0.26% w/w of compounds I-IV relative to the amount of tasquinimod. Therefore, according to the selected criterion for approval, the batch of capsules was determined to be suitable for distribution.

### Example 21

### Assessment of Oral Capsules Containing 0.25 mg of Tasquinimod

The batch of capsules manufactured in Example 19 was assessed by a method as described in Example 20, except for keeping the blister pack with the capsules at 25° C./60% RH for 6 months, whereby the criterion for approving the batch was that the capsules of the sample, at the end of the 6-month stability testing, contain a total amount of compounds I-IV of not more than 5.0% w/w, relative to the amount of tasquinimod in the capsules. At the end of the 6-month stability testing, the capsules contained 0.08% w/w of compound I, less than 0.05% w/w of compound II, 0.16% w/w of compound III, and less than 0.05% w/w of compound IV, relative to the amount of tasquinimod (0.243 mg/capsule), viz. in total less than 0.34% w/w of compounds I-IV relative to the amount of tasquinimod. Therefore, according to the selected criterion for approval, the batch of capsules was determined to be suitable for distribution.

### Example 22

### Assessment of Oral Capsules Containing 0.25 mg of Tasquinimod

The batch of capsules manufactured in Example 19 was assessed by a method as described in Example 20, except for keeping the blister pack with the capsules at 25° C./60% RH for 9 months, whereby the criterion for approving the batch was that the capsules of the sample, at the end of the 9-month stability testing, contain a total amount of compounds I-IV of not more than 5.0% w/w, relative to the amount of tasquinimod in the capsules. At the end of the 9-month stability testing, the capsules contained 0.12% w/w of compound I, less than 0.05% w/w of compound II, 0.21% w/w of compound III, and less than 0.05% w/w of compound IV, relative to the amount of tasquinimod (determined to be 0.243 mg/capsule), viz. in total less than 0.43% w/w of compounds I-IV relative to the amount of tasquinimod. Therefore, according to the selected criterion for approval, the batch of capsules was determined to be suitable for distribution.

### Example 23

### Assessment of Oral Capsules Containing 0.25 mg of Tasquinimod

The batch of capsules manufactured in Example 19 was assessed by a method as described in Example 20, except for keeping the blister pack with the capsules at 25° C./60% RH for 12 months, whereby the criterion for approving the batch was that the capsules of the sample, at the end of the 12-month stability testing, contain a total amount of compounds I-IV of not more than 5.0% w/w, relative to the amount of tasquinimod in the capsules. At the end of the 12-month stability testing, the capsules contained 0.11% w/w of compound I, less than 0.05% w/w of compound II, 0.26% w/w of compound III, and less than 0.05% w/w of compound IV, relative to the amount of tasquinimod (determined to be 0.245 mg/capsule), viz. in total less than 0.47% w/w of compounds I-IV relative to the amount of tasquinimod. Therefore, according to the selected criterion for approval, the batch of capsules was determined to be suitable for distribution.

### Example 24

### Assessment of Oral Capsules Containing 0.25 mg of Tasquinimod

The batch of capsules manufactured in Example 19 was assessed by a method as described in Example 20, except for keeping the blister pack with the capsules at 25° C./60% RH for 18 months, whereby the criterion for approving the batch was that the capsules of the sample, at the end of the 18-month stability testing, contain a total amount of compounds I-IV of not more than 5.0% w/w, relative to the amount of tasquinimod in the capsules. At the end of the 18-month stability testing, the capsules contained 0.10% w/w of compound I, 0.05% w/w of compound II, 0.35% w/w of compound III, and 0.07% w/w of compound IV, relative to the amount of tasquinimod (determined to be 0.245 mg/capsule), viz. in total 0.57% w/w of compounds I-IV relative to the amount of tasquinimod. Therefore, according to the selected criterion for approval, the batch of capsules was determined to be suitable for distribution.

### Example 25

### Assessment of Oral Capsules Containing 0.25 mg of Tasquinimod

The batch of capsules manufactured in Example 19 was assessed by a method as described in Example 20, except for keeping the blister pack with the capsules at 25° C./60% RH for 24 months, whereby the criterion for approving the batch was that the capsules of the sample, at the end of the 24-month stability testing, contain a total amount of compounds I-IV of not more than 5.0% w/w, relative to the amount of tasquinimod in the capsules. At the end of the 24-month stability testing, the capsules contained 0.13% w/w of compound 1, 0.05% w/w of compound II, 0.41% w/w of compound III, and 0.10% w/w of compound IV, relative to the amount of tasquinimod (determined to be 0.237 mg/capsule), viz. in total 0.69% w/w of compounds I-IV relative to the amount of tasquinimod. Therefore, according to the selected criterion for approval, the batch of capsules was determined to be suitable for distribution.

### Example 26

### Assessment of Oral Capsules Containing 0.25 mg of Tasquinimod

The batch of capsules manufactured in Example 19 was assessed by a method as described in Example 20, except for keeping the blister pack with the capsules at 25° C./60% RH for 36 months, whereby the criterion for approving the batch was that the capsules of the sample, at the end of the 36-month stability testing, contain a total amount of compounds I-IV of not more than 5.0% w/w, relative to the amount of tasquinimod in the capsules. At the end of the 36-month stability testing, the capsules contained 0.16% w/w of compound 1, 0.06% w/w of compound II, 0.57% w/w of compound III, and 0.15% w/w of compound IV, relative to the amount of tasquinimod (determined to be 0.237 mg/capsule), viz. in total 0.94% w/w of compounds I-IV relative to the amount of tasquinimod. Therefore, according to the selected criterion for approval, the batch of capsules was determined to be suitable for distribution.

### Example 27

### Assessment of Oral Capsules Containing 0.25 mg of Tasquinimod

The batch of capsules manufactured in Example 19 was assessed by a method as described in Example 20, except for keeping the blister pack with the capsules at 25° C./60% RH for 48 months, whereby the criterion for approving the batch was that the capsules of the sample, at the end of the 48-month stability testing, contain a total amount of compounds I-IV of not more than 5.0% w/w, relative to the amount of tasquinimod in the capsules. At the end of the 48-month stability testing, the capsules contained 0.16% w/w of compound I, 0.06% w/w of compound II, 0.79% w/w of compound III, and 0.19% w/w of compound IV, relative to the amount of tasquinimod (determined to be 0.238 mg/capsule), viz. in total 1.2% w/w of compounds I-IV relative to the amount of tasquinimod. Therefore, according to the selected criterion for approval, the batch of capsules was determined to be suitable for distribution.

### Example 28

### Manufacture and Multiple Assessments of Oral Capsules Containing 0.5 mg of Tasquinimod

A batch of oral capsules (200000 capsules) having a target dose strength of 0.5 mg of tasquinimod/capsule was manufactured according to the procedure described in Example 19. The capsules were conventional, solid, immediate release capsules (white, size 4 hard gelatin shell capsules), each capsule containing 150 mg of a powder composed of tasquinimod (target weight 0.5 mg), pregelatinised starch from maize (Starch 1500®) (146.5 mg) and hydrogenated vegetable oil (Lubritab®) (3.00 mg).

The obtained batch of capsules was subjected to multiple assessments, viz. a first assessment comprising determining the amount of tasquinimod and the amount of compounds I, II, III and IV in a sample of the capsules directly after manufacturing, whereby the selected criterion for approving the batch for distribution was that the capsules of the sample, in a water impermeable blister pack, contain not more than in total 5% w/w of compounds I-IV relative to the amount of tasquinimod, followed by eight further, independent assessments, by determining the amount of tasquinimod and the amount of each one of compounds I, II, III and IV in a sample of the capsules at the end of a period of stability testing (by keeping the capsules, in a water impermeable blister pack, at 25° C./60% RH) of 3, 6, 9, 12, 18, 24, 36 and 48 months, respectively, whereby each time the criterion for approving the batch was that the capsules of the sample, at the end of each period of stability testing, contain a total amount of compounds I-IV of not more than 5.0% w/w, relative to the amount of tasquinimod in the capsules. Table 3 shows the determined amounts of compounds I-IV and tasquinimod in the capsules at after manufacturing (viz. at 0 months) and at the end of each period of stability testing.

TABLE 3

| | Stability testing (months) | | | | | | | | |
|---|---|---|---|---|---|---|---|---|---|
| Test | 0 | 3 | 6 | 9 | 12 | 18 | 24 | 36 | 48 |
| Tasquinimod | 0.500 | 0.501 | 0.497 | 0.504 | 0.500 | 0.501 | 0.486 | 0.490 | 0.494 |
| (mg/capsule) | | | | | | | | | |
| Decomposition | | | | | | | | | |
| products | | | | | | | | | |
| (% w/w)* | | | | | | | | | |
| Compound I | <0.05 | <0.05 | 0.05 | 0.06 | 0.07 | 0.06 | 0.07 | 0.07 | 0.08 |
| Compound II | <0.05 | <0.05 | <0.05 | <0.05 | <0.05 | <0.05 | <0.05 | <0.05 | <0.05 |
| Compound III | <0.05 | 0.05 | 0.09 | 0.11 | 0.13 | 0.16 | 0.19 | 0.27 | 0.36 |
| Compound IV | <0.05 | <0.05 | <0.05 | <0.05 | <0.05 | <0.05 | 0.06 | 0.09 | 0.11 |

*relative to the amount of tasquinimod in capsule

The total amount of compounds I-IV was below 5% w/w relative to the amount of tasquinimod in the capsules of each sample and therefore according to each assessment the batch of capsules was determined as suitable for distribution.

### Example 29

### Manufacture and Multiple Assessments of Oral Capsules Containing 1.0 mg of Tasquinimod

A batch of oral capsules (200000 capsules) having a target dose strength of 1.0 mg of tasquinimod/capsule was manufactured according to the procedure described in Example 19. The capsules were conventional, solid, immediate release capsules (white, size 4 hard gelatin shell capsules), each capsule containing 150 mg of a powder composed of tasquinimod (target weight 1.0 mg), pregelatinised starch from maize (Starch 1500®) (146.0 mg) and hydrogenated vegetable oil (Lubritab®) (3.00 mg).

The obtained batch of capsules was subjected to multiple assessments, as described in Example 28, applying the same criteria for approval of the capsules for distribution. Table 4 shows the determined amounts of compounds I-IV and tasquinimod in the capsules at after manufacturing (viz. at 0 months) and at the end of each period of stability testing.

TABLE 4

| | Stability testing (months) | | | | | | | | |
|---|---|---|---|---|---|---|---|---|---|
| Test | 0 | 3 | 6 | 9 | 12 | 18 | 24 | 36 | 48 |
| Tasquinimod | 0.992 | 0.989 | 0.988 | 1.007 | 0.992 | 0.982 | 0.997 | 1.000 | 0.987 |
| (mg/capsule) | | | | | | | | | |
| Decomposition | | | | | | | | | |
| products | | | | | | | | | |
| (% w/w)* | | | | | | | | | |
| Compound I | <0.05 | <0.05 | <0.05 | <0.05 | <0.05 | <0.05 | <0.05 | <0.05 | <0.05 |
| Compound II | <0.05 | <0.05 | <0.05 | 0.06 | 0.06 | 0.06 | 0.06 | 0.07 | 0.08 |
| Compound III | <0.05 | <0.05 | <0.05 | <0.05 | 0.06 | 0.09 | 0.10 | 0.15 | 0.18 |
| Compound IV | <0.05 | <0.05 | <0.05 | <0.05 | <0.05 | <0.05 | <0.05 | <0.05 | <0.05 |

*relative to the amount of tasquinimod in capsule

The total amount of compounds I-IV was below 5% w/w relative to the amount of tasquinimod in the capsules of each sample and therefore according to each assessment the batch of capsules was determined as suitable for distribution.

### Examples 30-46

Examples 30-46 are identical with Examples 2-18, respectively, except that the criterion for approval was a maximum amount of compound III of less than 0.5% w/w relative to the amount of tasquinimod. According to the criterion, the capsules were determined as suitable for distribution.

### Examples 47-57

Examples 47-57 are identical with Examples 19-29, respectively, except that the criterion for approval was a maximum total amount of decomposition product of less than 2.0% w/w relative to the amount of tasquinimod. According to the criterion, the capsules were determined as suitable for distribution.

### Examples 58-68

Examples 58-68 are identical with Examples 47-57, respectively, except that the criterion for approval was additionally a maximum amount of each one of compounds I-IV of less than 0.5% w/w relative to the amount of tasquinimod. According to the criterion, the capsules were determined as suitable for distribution.

1. A pharmaceutical composition comprising a therapeutically effective amount of tasquinimod, or a pharmaceutically acceptable salt thereof, and one or more pharmaceutically acceptable excipients, said pharmaceutical composition further comprising one or more compounds selected from: 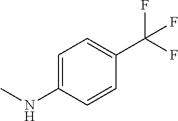
N-methyl-4-(trifluoromethyl)aniline, 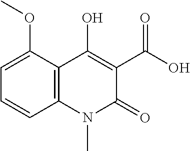 [SMILES: COc1cccc2c1c(O)c(C(=O)O)c(=O)n2C]
4-hydroxy-5-methoxy-1-methyl-2-oxo-1,2-dihydroquinoline-3-carboxylic acid, 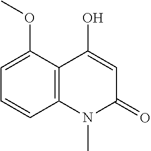
4-hydroxy-5-methoxy-1-methylquinoline-2(1H)-one, and 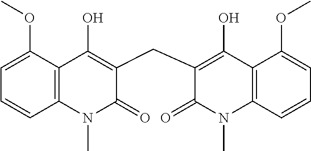 [SMILES: COc1cccc2c1c(O)c(Cc1c(O)c3c(OC)cccc3n(C)c1=O)c(=O)n2C]
3,3′-methylenebis(4-hydroxy-5-methoxy-1-methylquinoline-2(1H)-one),
and pharmaceutically acceptable salts thereof.

2. The pharmaceutical composition of claim 1, comprising said one or more compounds in a total amount of no more than 5% w/w, relative to the amount of tasquinimod in the composition.

3. The pharmaceutical composition of claim 2, wherein the total amount is no more than 2% w/w, relative to the amount of tasquinimod in the composition.

4. The pharmaceutical composition of claim 1, comprising an amount of no more than 2% w/w of any one of said one or more compounds, relative to the amount of tasquinimod in the composition.

5. The pharmaceutical composition claim 4, wherein the amount of any one of said one or more compounds is no more than 0.5% w/w, relative to the amount of tasquinimod in the composition.

6. A pharmaceutical dose unit for oral administration, comprising the pharmaceutical composition of claim 1.

7. The pharmaceutical dose unit of claim 6, which is a capsule or tablet.

8.-10. (canceled)

11. A method for assessing a pharmaceutical product containing a therapeutically effective amount of tasquinimod or a pharmaceutically acceptable salt thereof, and one or more pharmaceutically acceptable excipients, by obtaining a sample of said product and determining the amount of a tasquinimod decomposition product in said sample, said tasquinimod decomposition product comprising one or more compounds selected from N-methyl-4-(trifluoromethyl)aniline, 4-hydroxy-5-methoxy-1-methyl-2-oxo-1,2-dihydroquinoline-3-carboxylic acid, 4-hydroxy-5-methoxy-1-methylquinoline-2(1H)-one, and 3,3′-methylenebis(4-hydroxy-5-methoxy-1-methylquinoline-2(1H)-one), and pharmaceutically acceptable salts thereof.)

12. The method of claim 11, comprising keeping the sample at a temperature of 20° C. to 40° C., and a relative humidity of 30% to 75%, for a time period of from 2 weeks to 5 years before determining the amount of the tasquinimod decomposition product in said sample.

13. The method of claim 11, for determining whether a pharmaceutical product is suitable for distribution, wherein the pharmaceutical product is determined as suitable for distribution only if the sample contains not more than 5% w/w of the decomposition product, relative to the amount of tasquinimod in the sample.

14. The method of claim 13, wherein the pharmaceutical product is determined as suitable for distribution only if the sample contains not more than 2% w/w of the decomposition product, relative to the amount of tasquinimod in the sample.

15. The method of claim 11, for determining whether a pharmaceutical product is suitable for distribution, wherein the pharmaceutical product is determined as suitable for distribution only if the sample contains not more than 2% w/w of any one of said compounds, relative to the amount of tasquinimod in the sample

16. The method of claim 15, wherein the pharmaceutical product is determined as suitable for distribution only if the sample contains not more than 0.5% w/w of any one of said compounds, relative to the amount of tasquinimod in the sample.

17. A process for the manufacture of a pharmaceutical product comprising a pharmaceutically effective amount of tasquinimod or a pharmaceutically acceptable salt thereof, and a pharmaceutically acceptable excipient, said pharmaceutical product further comprising one or more compounds selected from N-methyl-4-(trifluoromethyl)aniline, 4-hydroxy-5-methoxy-1-methyl-2-oxo-1,2-dihydroquinoline-3-carboxylic acid, 4-hydroxy-5-methoxy-1-methylquinoline-2(1H)-one, and 3,3′-methylenebis(4-hydroxy-5-methoxy-1-methylquinoline-2(1H)-one), said process comprising preparing a pharmaceutical composition comprising a pharmaceutically effective amount of tasquinimod or a pharmaceutically acceptable salt thereof and one or more pharmaceutically acceptable excipients, optionally processing the composition to obtain a pharmaceutical dose unit, and assessing the pharmaceutical product by a method as defined in claim 11.

18. (canceled)

19. The compound 3,3′-methylenebis(4-hydroxy-5-methoxy-1-methylquinoline-2(1H)-one), or a pharmaceutically acceptable salt thereof.

20. (canceled)

21. A method for the treatment of cancer, by administering a pharmaceutical composition as defined in claim 1, to a mammal in need of such treatment.

22. The method of claim 21, wherein the cancer is a hematologic cancer, or a solid cancer.

23. The method of claim 22, wherein the hematological cancer is selected from multiple myeloma, lymphoma, myelodysplastic syndrome, myeloproliferative neoplasm, and leukemia, and the solid cancer is selected from bladder cancer, melanoma, lung cancer, colorectal cancer, breast cancer, pancreatic cancer, prostate cancer, renal cell carcinoma, ovarian cancer, neuroendocrine tumors (NET) and gastroenteropancreatic neuroendocrine tumors (GEPNET).
